(19) United States
(12) Patent Application Publication
(10) Pub. No.: US 2025/0049968 A1
(43) Pub. Date: Feb. 13, 2025

(54) THERAPEUTIC AND IMAGING AGENTS FOR TARGETING MYOCARDIAL TISSUE

(71) Applicant: The Board of Regents of the University of Oklahoma, Norman, OK (US)

(72) Inventors: Venkateswararao Eeda, Oklahoma City, OK (US); Vibhudutta Awasthi, Edmond, OK (US)

(73) Assignee: The Board of Regents of the University of Oklahoma, Norman, OK (US)

(21) Appl. No.: 18/921,938

(22) Filed: Oct. 21, 2024

Related U.S. Application Data
(63) Continuation-in-part of application No. PCT/US2023/019194, filed on Apr. 20, 2023.

(51) Int. Cl. A61K 51/04; A61K 9/127; A61K 47/54; A61K 47/60; A61P 9/00

(52) U.S. Cl. CPC A61K 51/0474; A61K 9/127; A61K 47/545; A61K 47/60; A61P 9/00; A61K 2123/00

(57) ABSTRACT

Analogs and derivatives of omecamtiv mecarbil (OM), including an ¹⁸F-labeled analog, and methods of synthesis thereof. Cardiac myosin targeting vectors comprising a lipid anchoring/solubilizing moiety conjugated to a head made of OM or the OM analog or derivative. Liposomes comprising a cardiac-treating cargo molecule and the cardiac myosin targeting vector. Methods of treating a cardiac condition or disease in a subject by administering to the subject the cardiac-treating cargo molecule and the cardiac myosin targeting vector. Methods of synthesizing an ¹⁸F-labeled analog of OM.

### CROSS-REFERENCE TO RELATED APPLICATIONS

This patent application is a continuation-in-part of PCT Patent Application No. PCT/US2023/019014, filed on Apr. 20, 2023, which claims priority to U.S. Provisional Application No. 63/333,380 filed on Apr. 21, 2022, the entire contents of both of which are hereby expressly incoporated herein by reference.

### BACKGROUND

Myocardial infarction (MI) is the leading cause of death in most developed nations throughout the world. About 735,000 individuals suffer MI every year in the USA. It is the most common form of coronary heart disease (CHD). One American dies due to MI every 60 seconds. MI occurs when a coronary artery is occluded, which reduces the blood flow to the downstream myocardial tissue and creates oxygen and nutritional deficit. Many different classes of drugs, including thrombolytics, antiplatelets, antiarrhythmics, analgesics, vasodilators, and cardiac depressants, have been used for MI patients, but most of these do not directly address the recovery processes of damaged myocardium.

Cardioprotective therapies that can reduce the growth of infarct, overcome inflammatory reperfusion injury, and promote recovery are considered to have translational potential. To salvage the dying tissue, experimental approaches involve the supply of growth factors, cytokines, drugs, and other biomolecules to the cardiomyocytes. A few reports also exist for cardiac gene therapy using intravenously injected adeno-associated viral vectors, ultrasound-targeted microbubble destruction, catheter-based gene delivery and MI-specific targeted peptide conjugation. However, drug delivery to the heart has remained a highly challenging proposition because of the constant dynamic cycles of the heart and massive exchanges of blood volume. These unique features do not permit administered therapeutics to stay inside the heart very long, which severe limits the duration of exposure of the drug to the heart tissue. Thus, preferential delivery of a drug to the myocardial tissue frequently involves invasive surgical procedures involving opening the chest wall. Although intra-myocardial or epicardial injections can be considered non-surgical approaches of accessing heart tissue, open surgery is indispensable for accurate drug targeting. A liposome-based technology for targeted delivery to the heart tissue by intravenous administration would be a significant advance in the treatment of heart disease.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic depiction of the general process of the disclosure in which cargo-loaded liposomes bearing target-specific vectors are provided which bind to target molecules on cardiac tissue and release the cargo (represented as triangles) at the cardiac tissue.

FIG. 2 depicts a liposome comprising two drug-bearing zones and decorated with a targeting vector constructed of omecamtiv mecarbil (OM) linked via a polyethylene (PEG) linker to a distearoylphosphatidylethanolamine (DSPE) molecule which anchors the targeting vector within the liposome.

FIGS. 3A and 3B shows nuclear gamma camera images of control mice and mice with myocardial infarct (MI) injected with ^{99m}Tc-labeled OM-liposomes after 15 minutes (A) and 120 minutes (B).

FIGS. 4A, 4B and 4C show a whole-body nuclear gamma camera image of a mouse with myocardial infarct (MI) showing significant accumulation of ^{99m}Tc-OM-liposomes in the heart (A). This image was acquired 3 h after injection. As expected of particulate carriers, significant uptake in liver and bladder was observed. A representative set of ex vivo images of MI heart and sham heart after 24 h of ^{99m}Tc-OM-liposome injection is shown in (B). The MI heart showed higher uptake as compared to the sham heart. Radioactive counts of ^{99m}Tc-OM-liposomes associated with sham hearts (n=4) versus MI (n=3) mice are shown in (C).

FIG. 5A shows results when cardiac-targeted OM-liposomes were labeled with Texas red dye and injected into control mice. 4 h after 0.05 mL injection, mice were killed to excise heart and process for fluorescence microscopy.

FIG. 5B shows results when cardiac-targeted OM-liposomes were labeled with Texas red dye and injected into mice with MI. After 4 h of 0.05 mL injection, mice were killed to excise heart and process for fluorescence microscopy. MI tissues showed more generalized red fluorescence as compared to the control tissues in FIG. 5A.

FIG. 6 shows a whole-body PET of ¹⁸F-OM in mice. Approximately 80 uCi of ¹⁸F-OM was injected and images were acquired after 4 h. There was no visible accumulation of ¹⁸F-OM in normal hearts of control mice (left panel), but MI mice (right panel) showed a high accumulation of ¹⁸F-OM (arrow).

FIG. 7 shows cardiac uptake of [18F]-OMA normal (left), and MI animal (right). The MI animal shows distinctly high uptake of [18F]-OMA in comparison to the normal (control) animal.

FIG. 8 shows result of ¹⁸F-fluoride dynamic PET/CT: After 2 h, 68 μCi/50 μL, iv of labeled compound to a normal (control) mouse (left side of top panel). After 2 h, 68 μCi/51μ, iv of labeled compound to myocardial injury (MI) mouse (right side of top panel). As shown in the bottom panel (work flow of imaging), animals were anesthetized and injected the tracer via a tail vein the anterior descending branch of the left coronary artery was ligated to create MI in mice, followed by reperfusion after 20 min to induce dysfunctional myocardium. After 1 h of reperfusion animals were subjected to CT/PET imaging, and after 4 h animals were sacrificed for bio-distribution analysis.

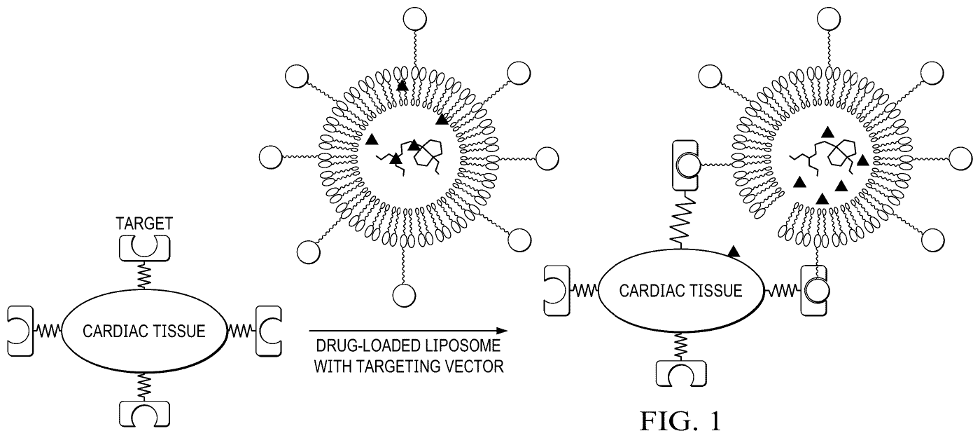

### DETAILED DESCRIPTION

Targeted liposomes (liposomal carriers) are attractive delivery vehicles as they are injected intravenously and circulate in blood for long periods. The present disclosure is directed to novel targeting vectors and liposomal formulations comprising the targeting vectors which actively target cardiac tissue, thus enabling delivery of therapeutic drugs or imaging agents directly to the heart. In one non-limiting embodiment the vesicular surface of the cargo-loaded liposomes is decorated with the targeting vectors which comprise a cardiac myosin-binding compound to make cardiac-targeted liposomes. The targeting vector comprises a cardiac myosin targeting molecule (the “head”) which is linked to an anchoring/solubilizing moiety (the “anchor”) via a linker molecule (the “linker”). In certain embodiments the cardiac myosin targeting molecule, or head, is omecamtiv mecarbil, previously known as CK-1827452, or analogs or derivatives thereof which bind to cardiac myosin.

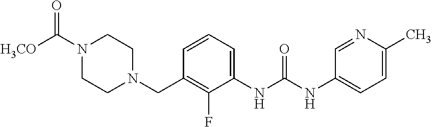 [SMILES: COC(=O)N1CCN(Cc2cccc(NC(=O)Nc3ccc(C)nc3)c2F)CC1]

Omecamtiv mecarbil (OM) is a selective and direct activator of cardiac myosin. The targeting vector therefore comprises in at least certain embodiments, OM, or an analog or derivative thereof, linked to the anchor (the anchoring/solubilizing moiety) via the linker, forming a head-linker-anchor (or OM-linker-anchor) conjugate. The anchor, which is generally a lipid, facilitates anchoring of the targeting vector in the liposome bilayer and/or in the serum in isolation. For example, the linker may be a poly(ethylene glycol) (PEG) linker and the anchor (the anchor/solubilizing moiety) may be, in non-limiting embodiments, a phospholipid such as a fatty acid phosphatidylethanolamine, fatty acid phosphatidylserine, or fatty acid phosphatidylcholine. In alternative embodiments, a therapeutic agent or an imaging agent may be conjugated to the head for “as-is” delivery to the cardiac tissue. Its binding to myosin increases contractile force of cardiac myocytes without altering intracellular calcium and oxygen consumption. OM allosterically activates the S1 catalytic domain of myosin and increases the number of myosin heads primed for engagement with actin filaments during systole. Clinical studies have demonstrated that OM improves left ventricular systolic function, as reflected by increased systolic ejection time and ejection fraction in healthy subjects and patients. It exhibits a linear dose-proportional pharmacokinetics (PK) profile with a median time (tₘₐₓ) of maximum observed plasma concentration (Cₘₐₓ) of 2 h and a mean apparent terminal elimination half-life (t_{1/2}) of 18.5 h. OM is primarily metabolized in humans by the cytochrome P450 (CYP) enzymes CYP3A4 and CYP2D6 and converted into M3 and M4 metabolites, which are less potent than OM. Hepatic impairment, renal impairment, or hemodialysis does not significantly affect the PK profile of OM.

The clinical goal of promptly restoring blood supply in acute MI patients is accomplished by the percutaneous coronary intervention (PCI), or thrombolytic therapy. However, reperfusion can cause paradoxical damage by a phenomenon called ischemia-reperfusion injury (IRI). IRI is associated with mitochondrial dysfunction, increase in reactive oxygen species, and hypercontractility, culminating in the loss of viable myocardium as the cardiomyocytes fail to readjust to the aerobic metabolism. It is estimated that close to 50% of the final myocardial infarct size in recanalized patients is due to IRI. Therefore, post-reperfusion cardioprotection is the major unmet challenge in MI management. Initially, cardioprotective strategies evolved individually addressing isolated mechanisms such as redox stress, inflammation, vasodilation, hypothermia, metabolic modulation, reperfusion injury salvage kinase (RISK) activation, mitochondrial permeability transition pore (MPTP) inhibition, etc., but highly disappointing translational outcomes has now led to a realization that a more effective approach may be to simultaneously target more than one factor at a time. Such combined pharmacologic treatment is possible if two or more drug candidates modulating intracellular Ca, inhibit MPTP, and activate RISK are simultaneously delivered to the cardiac tissue by OM-liposomes.

Therapeutic agents and diagnostic agents that may be loaded into the OM-liposomes of the present disclosure, or which may be carried by the targeting vectors alone, include but are not limited to the various active agents described elsewhere herein. For example, in various embodiments, the presently disclosed cardiac-targeted liposomes can be used to deliver all classes of cardiovascular drugs, including biologics such as RNA, DNA, antibodies and antibody fragments, peptides, and proteins, as well as small molecules such as cardioprotective agents for use in treating in acute MI patients. The OM-liposomes may comprise or be loaded, as noted above, with imaging agents, including radionuclides, for use in diagnostic and imaging procedures.

In certain embodiments, the presently disclosed technology can be employed to modify non-viral as well as viral gene delivery vehicles, i.e., by inserting the head-linker-anchor (e.g., OM-PEG-DSPE, as described below) into the non-viral or viral vector which contains the genetic information. The gold standard for cardiomyocyte gene transfer is parvovirus adeno-associated virus (AAV). Important variables affecting overall efficacy of cardiac gene therapy are the route of administration and the extent of gene delivery. A most common and effective technique for cardiac gene transfer is direct intramyocardial injection. This can be either through complex, risky, and invasive surgical access by mini-thoracotomy followed by transepicardial delivery, or by intraventricular injection using a percutaneous catheter to achieve transendocardial delivery. Insertion of the cardiac-targeting moieties of the present disclosure into the AAV envelope to achieve re-targeting and cardiotropism by simple intravenous delivery is one non-limiting embodiment of the present disclosure.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, published patent applications, and non-patent publications including published articles mentioned in the specification or referenced in any portion of this application, including U.S. Provisional Application No. 63/333,380 filed on Apr. 21, 2022, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. A range is intended to include any sub-range therein, although that sub-range may not be explicitly designated herein. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 2-125 therefore includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, and 125, as well as sub-ranges within the greater range, e.g., for 2-125, sub-ranges include but are not limited to 2-50, 5-50, 10-60, 5-45, 15-60, 10-40, 15-30, 2-85, 5-85, 20-75, 5-70, 10-70, 28-70, 14-56, 2-100, 5-100, 10-100, 5-90, 15-100, 10-75, 5-40, 2-105, 5-105, 100-95, 4-78, 15-65, 18-88, and 12-56. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

The terms “increase,” “increasing,” “enhancing,” or “enhancement” are defined as indicating a result that is greater in magnitude than a control number derived from analysis of a cohort, for example, the result can be a positive change of at least 5%, 10%, 20%, 30%, 40%, 50%, 80%, 100%, 200%, 300% or even more in comparison with the control number. Similarly, the terms “decrease,” “decreasing,” “lessening,” or “reduction” are defined as indicating a result that is lesser in magnitude than a control number, for example, the result can be a negative change of at least 5%, 10%, 20%, 30%, 40%, 50%, 80%, 100%, 200%, 300% or even more in comparison with the control number.

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±25%, or ±20%, or ±10%, or ±5%, or 1%, or ±0.10% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

The term “active agent” as used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals or subjects within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The term “ameliorate” means a detectable or measurable improvement in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

Where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Where used herein, the pronoun “we” is intended to refer to all persons involved in a particular aspect of the work disclosed herein and as such may include non-inventor laboratory assistants and collaborators working under the supervision of the inventors.

The active agents of the present disclosure can be combined into formulations or treatments that are synergistic. As used herein the terms “synergism,” “synergistic,” or “synergistic effect” refers to a therapeutic effect or result that is greater than the additive effects of each active agent used individually. Presence or absence of a synergistic effect for a particular combination of treatment substances can be quantified by using the Combination Index (CI) (e.g., Chou, Pharmacol Rev, 2006. 58(3): 621-81), wherein CI values lower than 1 indicate synergy and values greater than 1 imply antagonism. Combinations of the inhibitors and antagonists of the present disclosure can be tested in vitro for synergistic cell growth inhibition using standard cell lines for particular cancers, or in vivo using standard animal cancer models. A synergistic effect of a combination described herein can permit, in some embodiments, the use of lower dosages of one or more of the components of the combination. A synergistic effect can also permit, in some embodiments, less frequent administration of at least one of the administered active agents. Such lower dosages and reduced frequency of administration can reduce the toxicity associated with the administration of at least one of the therapies to a subject without reducing the efficacy of the treatment.

The term “coadministration” refers to administration of two or more active agents, e.g., a cardiac-targeted composition as described herein and another active agent. The timing of coadministration depends in part of the combination and compositions administered and can include administration at the same time, just prior to, or just after the administration of one or more additional therapies Coadministration is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating MI, and co-occurring conditions thereof.

In at least certain compounds of the present disclosure, polyethylene glycol (PEG) molecules (also known as poly(ethylene oxide) and poly(oxyethylene)) are used, for example as linkers to link other compounds together to for drug conjugates. PEG comprises repeating units of ethylene glycol, and is available in different average molecular weights (MW) based on the average number of ethylene glycol units in the PEG molecules of the particular PEG composition. For example, PEG₈₈, a PEG molecule with 2 ethylene glycol units, has a MW of 88 Daltons (Da). PEG₄₀₀, a PEG molecule with about 8 ethylene glycol units, has a MW of 400 Daltons (Da). PEG_{60,000}, a PEG molecule with about 1364 ethylene glycol units, has a MW of about 60,000. The PEG molecule may comprise up to 30,000 ethylene glycol units, Other examples include, but are not limited to, PEG₂₀₀ having an average MW of about 200 Daltons (Da), PEG₃₀₀ having an average MW of about 300 Da, PEG₄₀₀ having an average MW of about 400 Da, PEG₅₀₀ having an average MW of about 500 Da, PEG₇₅₀ having an average MW of about 750 Da, PEG₁₀₀₀ having an average MW of about 1000 Da, PEG₁₅₀₀ having an average MW of about 1500 Da, PEG₂₀₀₀ having an average MW of about 2000 Da, PEG₃₀₀₀ having an average MW of about 3000 Da, PEG₃₃₅₀ having an average MW of about 3350 Da, PEG₃₅₀₀ having an average MW of about 3500 Da, PEG₄₀₀₀ having an average MW of about 4000 Da, PEG₅₀₀₀ having an average MW of about 5000 Da, PEG₆₀₀₀ having an average MW of about 6000 Da, PEG₇₅₀₀ having an average MW of about 7500 Da, PEG_{10,000} having an average MW of about 10,000 Da, PEG_{15,000} having an average MW of about 15,000 Da, PEG_{20,000} having an average MW of about 20,000 Da, PEG_{25,000} having an average MW of about 25,000 Da, PEG_{30,000} having an average MW of about 30,000 Da, PEG_{40,000} having an average MW of about 40,000 Da, PEG_{50,000} having an average MW of about 50,000 Da, and PEG_{60,000} having an average MW of about 60,000 Da. Where used herein the term PEG is intended to refer to any of the examples of PEG listed above, and to PEGs having MWs in the range of 88 and 60,000, unless a particular MW is specified. In other embodiments, the linker molecule may be an amino acid, a peptide, or a polypeptide,

In various non-limiting embodiments, the drug conjugates of the present disclosure include OM, or suitable derivatives thereof, which are linked via a linker (e.g., a PEG, amino acid, peptide or polypeptide) to an anchor-solubilizing moiety such as a phosphatidylethanolamine (PE). Examples of such PEs include but are not limited to distearoylphosphatidylethanolamine (DSPE), dipalmitoylphosphatidylethanolamine, dimyristoylphosphatidylethanolamine, diarachidylphosphatidylethanolamine, dilaurylphosphatidylethanolamine, dioleylphosphatidylethanolamine, palmitoylstearoylphosphatidylethanolamine, myristoylstearoylphosphatidylethanolamine, arachidylstearoylphosphatidylethanolamine, laurylstearoylphosphatidylethanolamine, oleylstearoylphosphatidylethanolamine, myristoylpalmitoylphosphatidylethanolamine, arachidylpalmitoylphosphatidylethanolamine, laurylpalmitoylphosphatidylethanolamine, arachidylmyristoylphosphatidylethanolamine, laurylmyristoylphosphatidylethanolamine, laurylarachidylphosphatidylethanolamine, oleylpalmitoylphosphatidylethanolamine, oleylmyristoylphosphatidylethanolamine, oleylarachidylphosphatidylethanolamine, and lauryloleylphosphatidylethanolamine. In other embodiments, anchoring/solubilizing moiety may comprise any one of the above moieties wherein the ethanolamine is substituted with seine (forming a phosphatidylserine (PS)) or choline (forming a phosphatidylcholine (PC)), such as distearoylphosphatidylserine or distearoylphosphatidylcholine. In other embodiments, the anchoring/solubilizing moiety may comprise a combination of two or more of the above moieties. In other embodiments, anchoring/solubilizing moiety may comprise a single saturated, unsaturated, or polyunsaturated lipid molecule comprising 2-28 carbon atoms, particularly 10-18 carbon atoms, such as a saturated, unsaturated, or polyunsaturated fatty acid. The anchor-solubilizing moiety may comprise a PE, PS or PC with a single fatty acid or two fatty acids, which may be selected from the group of saturated, unsaturated, and polyunsaturated fatty acids.

In particular, non-limiting examples, the targeting vector of the present disclosure is combined with a liposomal carrier in which a cargo molecule is disposed. In addition to other pharmaceutically acceptable carrier(s), the liposomal carrier may contain amphipathic agents such as lipids which exist in an aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, combinations thereof, and the like. Preparation of such liposomal formulations is well within the level of ordinary skill in the art, as disclosed, for example, in U.S. Pat. Nos. 4,235,871; 4,501,728; 4,837,028; and 4,737,323; the entire contents of each of which are incorporated herein by reference.

As used herein, the terms “liposome” and “liposomal carrier” are used interchangeably to refer to a vesicle composed of amphiphilic lipids arranged in a spherical bilayer or bilayers. Liposomes are unilamellar or multilamellar vesicles which have a membrane formed from a lipophilic material and an aqueous interior. The aqueous portion contains the active agent to be delivered. Liposomes can be made from phospholipids other than naturally-derived phosphatidylcholine. Neutral liposome compositions, for example, can be formed from dimyristoyl phosphatidylcholine (DMPC) or dipalmitoyl phosphatidylcholine (DPPC) or other similar lipids. Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example (but not by way of limitation), soybean PC, and egg PC. Another type is formed from mixtures of phospholipid and/or phosphatidylcholine and/or cholesterol.

The term “antibody” as used herein can refer to both intact “full length” antibodies as well as to antigen-binding fragments thereof (unless otherwise explicitly noted). The afore-mentioned antigen-binding fragments may also be referred to herein as antigen binding fragments, antigen binding compounds, antigen binding portions, binding fragments, binding portions, or antibody fragments. Also, as used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)₂ fragments, F(ab)₂ fragments, disulfide-linked Fvs (sdFv) (including bi-specific sdFvs), and anti-idiotypic (anti-Id) antibodies, dAb fragments, nanobodies, diabodies, triabodies, tetrabodies, linear antibodies, isolated CDRs, and epitope-binding fragments of any of the above. Regardless of structure, an antibody fragment refers to an isolated portion of the antibody that binds to the same antigen that is recognized by the intact antibody.

The antibodies of several embodiments provided herein may be monospecific, bispecific, trispecific, or of greater multispecificity, such as multispecific antibodies formed from antibody fragments. The term “antibody” also includes a diabody (homodimeric Fv fragment) or a minibody (V_{L}-V_{H}-C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, International Patent Application Publication Nos. WO 93/17715; WO 92/08802; WO 91/00360; and WO 92/05793; and U.S. Pat. Nos. 4,474,893; 4,714,681; 4,925,648; 5,573,920; and 5,601,819).

The term “monoclonal antibody” as used herein refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, monoclonal antibodies can be made by the hybridoma method first described by Kohler et al. (Nature, 256:495 (1975)), or may be made by recombinant DNA methods (see, for example, U.S. Pat. No. 4,816,567).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle(s) intended to facilitate its use. Interfering proteins and other contaminants can include, for example, cellular components of the cells from which an antibody is isolated or recombinantly produced. Sometimes monoclonal antibodies are at least 60%, at least 70%, at least 80%, at least 90%, at least 95%, or at least 99% w/w pure of interfering proteins and contaminants from production or purification. The antibodies and antigen binding compounds described herein can be provided in isolated and/or purified form.

In at least certain embodiments of the present disclosure, the term “therapeutic agent” refers to an active agent comprising an antibody and/or antibody-derived compound or other compound as described herein.

A “diagnostic agent,” which may also be referred to herein as an imaging agent, is a substance that is useful in diagnosing a disease or imaging a cell or tissue. Useful diagnostic agents of the present disclosure may include antibodies and antibody-derived compounds described herein, and may further comprise by linkage or other association radioisotopes, dyes, contrast agents, fluorescent compounds or molecules, and enhancing agents (e.g., paramagnetic ions).

An “immunoconjugate” or “antibody-drug conjugate” (ADC) is a conjugate of an antibody or antibody-derived compound with an atom, molecule, or a higher-ordered structure (e.g., with a liposome), a therapeutic agent, or a diagnostic agent.

As used herein, the term “antibody fusion protein” is a recombinantly produced antigen-binding molecule in which an antibody or antibody fragment is linked to another protein or peptide, such as the same or different antibody or antibody fragment. The fusion protein may comprise a single antibody component, a multivalent or multispecific combination of different antibody components, or multiple copies of the same antibody component, or other component described elsewhere herein. The fusion protein may additionally comprise an antibody or an antibody fragment and a therapeutic agent.

The basic structural unit of an antibody is a tetramer of subunits. Each tetramer includes two identical pairs of polypeptide chains, each pair having one “light” chain (about 25 kDa) and one “heavy” chain (about 50-70 kDa). The amino-terminal portion of each chain includes a variable region (variable heavy chain and variable light chain) of about 100 to 120 or more amino acids, which include portions called complementarity determining regions (CDRs) as described below, which are primarily responsible for antigen recognition. The three CDRs of the variable heavy chain may be referred to herein as CDRH1, CDRH2, and CDRH3. The three CDRs of the variable light chain may be referred to herein as CDRL1, CDRL2, and CDRL3. This variable region is initially expressed linked to a cleavable signal peptide. The variable region without the signal peptide is sometimes referred to as a mature variable region. Thus, for example, a “light chain mature variable region” means a light chain variable region without the light chain signal peptide. The carboxy-terminal portion of each chain defines a constant region primarily responsible for effector function. A CDR is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest.

Light chains are classified as either kappa or lambda. Heavy chains are classified as gamma, mu, alpha, delta, or epsilon, and define the antibody's isotype as IgG, IgM, IgA, IgD, and IgE, respectively. Within light and heavy chains, the variable and constant regions are joined by a “J” region of about 12 or more amino acids, with the heavy chain also including a “D” region of about 10 or more amino acids.

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR)joined by three hypervariable regions, which as noted above are known as CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3, and FR4.

The assignment of amino acids to each domain (FR1, CDR1, FR2, CDR2, FR3, CDR3, and FR4) is done in accordance with the protocols of the IMGT, e.g., see Ehrenmann, F., Kaas, Q. and Lefranc, M.-P., “IMGT/3Dstructure-DB and IMGT/DomainGapAlign: a database and tool for immunoglobulins or antibodies, T cell receptors, MHC, IgSF and MhcSF,” Nucl. Acids Res., 38(S1):D301-D307 (2010). DOI:10.1093/nar/kgp946. PMID:19900967, and Ehrenmann, F. and Lefranc, M.-P. Cold Spring Harb. Protocols, 2011(6):737-749. DOI:10.1101/pdb.prot5636. PMID:21632775.

In other embodiments, the assignment of amino acids to each domain may be done in accordance with the protocols of Kabat (Sequences of Proteins of Immunological Interest, National Institutes of Health, Bethesda, Md., 1987 and 1991), or Chothia & Lesk (J. Mol. Biol. 196:901-917 (1987); Chothia et al., Nature, 342:878-883 (1989)). Kabat also provides a widely used numbering convention (Kabat numbering) in which corresponding residues between different heavy chains or between different light chains are assigned the same number.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., “Epitope Mapping Protocols,” in Methods in Molecular Biology, Vol. 66, Glenn E. Morris, Ed. (1996).

Also within the scope of the present disclosure are antibodies or antibody-derived compounds thereof in which specific amino acids have been substituted, deleted, or added. These alternations do not have a substantial effect on the peptide's biological properties, such as (but not limited to) binding activity. For example, antibodies may have amino acid substitutions in the framework region, such as to improve binding to the antigen. In another example, a selected, small number of acceptor framework residues can be replaced by the corresponding donor amino acids. The donor framework can be a mature or germline human antibody framework sequence or a consensus sequence. Guidance concerning how to make phenotypically silent amino acid substitutions is provided in the following: Bowie et al. (Science, 247: 1306-1310 (1990)); Cunningham et al. (Science, 244: 1081-1085 (1989)); Ausubel (ed.) (Current Protocols in Molecular Biology, John Wiley and Sons, Inc. (1994)); Maniatis et al. (Molecular Cloning: A Laboratory Manual, Cold Spring Harbor laboratory, Cold Spring Harbor, N.Y. (1989)); Pearson (Methods Mol. Biol. 243:307-31 (1994)); and Gonnet et al. (Science, 256:1443-45 (1992)).

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent- (i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Compositions or methods “comprising” one or more recited elements may include other elements not specifically recited. For example, a composition that comprises an antibody may contain the antibody alone or in combination with other ingredients. The phrase “pharmaceutically acceptable salt” refers to pharmaceutically acceptable organic or inorganic salts of a presently-disclosed antibody, or binding fragment, or conjugate thereof, or agent administered with presently-disclosed antibody or fragment or conjugate thereof. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

A chimeric antibody is a molecule in which different portions are derived from different animal species. For example, an antibody may contain a variable region derived from a murine mAb and a human immunoglobulin constant region. Chimeric antibodies can be produced by recombinant DNA techniques, e.g., see Morrison et al. (Proc Natl Acad Sci, 81:6851-6855 (1984)). For example, a gene encoding a murine (or other species) monoclonal antibody molecule is digested with restriction enzymes to remove the region encoding the murine Fc, and the equivalent portion of a gene encoding a human Fc constant region is substituted. Chimeric antibodies can also be created by recombinant DNA techniques where DNA encoding murine variable regions can be ligated to DNA encoding the human constant regions, e.g., see International Patent Publication Nos. WO 87/002671 and WO 86/01533, and U.S. Pat. No. 4,816,567.

A chimeric antibody is a recombinant protein that contains the variable domains including the CDRs of an antibody derived from one species, for example a rodent or rabbit antibody, while the constant domains of the antibody molecule are generally derived from those of a human antibody. For veterinary applications, the constant domains of the chimeric antibody may be derived from that of other species, such as but not limited to, a cat, dog, or horse.

A chimeric antibody can be humanized by replacing the sequences of, for example, a murine FR in the variable domains of the chimeric antibody with one or more different human FR sequences. Specifically, mouse CDRs are transferred from heavy and light variable chains of the mouse immunoglobulin into the corresponding variable domains of a human antibody. As simply transferring mouse CDRs into human FRs may result in a reduction of antibody affinity, additional modifications might be required in order to restore the original affinity of the murine antibody. This can be accomplished by the replacement of one or more human residues in the FR regions with their murine counterparts to obtain an antibody with enhanced binding affinity to the D1R epitope or D2R epitope (e.g., see Tempest et al. (Biotechnology, 9:266 (1991)) and Verhoeyen et al. (Science, 239: 1534 (1988))). Techniques for producing humanized antibodies are known to persons having ordinary skill in the art and are disclosed, for example, by Jones et al. (Nature, 321: 522 (1986)), Riechmann et al. (Nature, 332: 323 (1988)), Verhoeyen et al. (Science, 239: 1534 (1988)), Carter et al. (Proc. Nat'l Acad. Sci. USA, 89: 4285 (1992)), Sandhu (Crit. Rev. Biotech. 12:437 (1992)), and Singer et al. (J. Immun. 150: 2844 (1993)).

As noted, an antibody's light chain variable region contains an FR interrupted by three different CDRs (CDRL1, CDRL2, and CDRL3), and a heavy chain region contains an FR interrupted by three different CDRs (CDRH1, CDRH2, and CDRH3). In one non-limiting embodiment, humanized antibodies are antibody molecules from non-human species having one, two, three, four, five or all six CDRs from the non-human species and a framework region from a human immunoglobulin molecule.

Ahumanized antibody is a genetically engineered antibody in which the variable heavy and variable light CDRs from a non-human “donor” antibody are grafted into human “acceptor” antibody sequences (see for example, U.S. Pat. Nos. 5,530,101; 5,585,089; 5,225,539; 6,407,213; 5,859,205; and 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two, and usually all three CDRs entirely or substantially from a donor antibody heavy chain, and a heavy chain variable region framework sequence, and heavy chain constant region, if present, substantially from human heavy chain variable region framework and constant region sequences. Similarly, a humanized light chain has at least one, two, and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain.

As noted, humanized antibodies can be generated by replacing framework sequences of the variable region that are not directly involved in antigen binding with equivalent sequences from human variable regions. Those methods include isolating, manipulating, and expressing the nucleic acid sequences that encode all or part of variable regions from at least one of a heavy or light chain. The recombinant DNA encoding the humanized antibody, or fragment thereof, can then be cloned into an appropriate expression vector. Each antibody light and heavy chain variable region consists of a framework region interrupted by the three corresponding CDRs. In one non-limiting embodiment, humanized antibodies are antibody molecules from non-human species having one, two, or all CDRs from the non-human species and a framework region from a human immunoglobulin molecule. Therefore, humanized antibodies can be generated by replacing framework sequences of the variable region that are not directly involved in antigen binding with equivalent sequences from human variable regions. Those methods include isolating, manipulating, and expressing the nucleic acid sequences that encode all or part of variable regions from at least one of a heavy or light chain. The recombinant DNA encoding the humanized antibody, or fragment thereof, can then be cloned into an appropriate expression vector.

The light and heavy chain variable regions can optionally be ligated to corresponding constant regions. CDR-grafted antibody molecules can be produced by CDR-grafting or CDR substitution. One, two, or all CDRs of an immunoglobulin chain can be replaced. For example, all of the CDRs of a particular antibody may be from at least a portion of a non-human animal (e.g., mouse, such as (but not limited to) CDRs shown herein), or only some of the CDRs may be replaced. It is only necessary to keep the CDRs which are required for specific and high binding affinity of the antibody to a target. Once expressed, antibodies can be purified according to standard procedures of the art, including but not limited to HPLC purification, column chromatography, and gel electrophoresis. Methods for producing human antibodies include, but are not limited to, those shown in U.S. Pat. Nos. 4,634,664; 4,634,666; 5,877,397; 5,874,299; 5,814,318; 5,789,650; 5,770,429; 5,661,016; 5,633,425; 5,625,126; 5,569,825; 5,545,806; 5,877,218; 5,871,907; 5,858,657; 5,837,242; 5,733,743; and 5,565,332; and International Patent Application Publication Nos. WO 91/17271; WO 92/01047; and WO93/12227.

A fully human antibody can be obtained from a transgenic non-human animal (see, e.g., Mendez et al. (Nature Genetics, 15: 146-156, 1997); and U.S. Pat. No. 5,633,425). Methods for producing fully human antibodies using either combinatorial approaches or transgenic animals transformed with human immunoglobulin loci are known in the art (e.g., Mancini et al. (New Microbiol., 27:315-28 (2004)); Conrad and Scheller (Comb. Chem. High Throughput Screen. 8:117-26 (2005)); and Brekke and Loset (Curr. Opin. Pharmacol., 3:544-50 (2003)). Such fully human antibodies are expected to exhibit even fewer side effects than chimeric or humanized antibodies and to function in vivo as essentially endogenous human antibodies. In certain non-limiting embodiments, the claimed methods and procedures may utilize human antibodies produced by such techniques.

A CDR in a humanized or human antibody may be defined as “substantially derived from” or “substantially identical to” a corresponding CDR in a non-human antibody when at least 60%, or at least 65%, or at least 70%, or at least 75%, or at least 80%, or at least 85%, or at least 90%, or at least 95%, or 100% of corresponding residues (as defined by Kabat) are identical between the respective CDRs. In some non-limiting embodiments, a CDR in a humanized antibody or human antibody is substantially derived from or substantially identical to a corresponding CDR in a non-human antibody when there are no more than one, two, or three conservative amino acid substitutions in any given CDR. The variable region framework sequences of an antibody chain or the constant region of an antibody chain are “substantially from” a human variable region framework sequence or human constant region, respectively, when at least about 60%, at least about 65%, at least about 70%, at least about 75%, at least about 80%, at least about 85%, at least about 90%, at least about 95%, or about 100% of corresponding residues (as defined by Kabat numbering) are identical. As noted elsewhere herein, although humanized antibodies often incorporate all six CDRs (e.g., as defined elsewhere herein) from a non-human (e.g., mouse or rabbit) antibody, they can also be made with less than all of the non-human CDRs (e.g., at least 2, 3, 4, or 5).

Heavy and light chain variable regions of humanized antibodies can be linked to at least a portion of a human constant region, for example, for human antibody isotypes IgG1, IgG2, IgG3, or IgG4. Light chain constant regions can be lambda or kappa. Antibodies can be expressed as, for example (but not by way of limitation): tetramers containing two light and two heavy chains, as separate heavy chains, light chains, as Fab, Fab′, F(ab′)₂, and Fv, or as single chain antibodies in which heavy and light chain variable domains are linked through a spacer. All antibody isotypes are encompassed by the present disclosure, including (but not limited to) IgG (e.g., IgG1, IgG2, IgG3, IgG4), IgM, IgA (IgA1, IgA2), IgD, or IgE. The antibodies or antigen-binding portions thereof may be mammalian (e.g., mouse, rabbit, human) antibodies or antigen-binding portions thereof.

Humanized or chimeric antibodies are typically produced by recombinant expression. Recombinant polynucleotide constructs typically include an expression control sequence operably linked to the coding sequences of antibody chains, including naturally-associated or heterologous promoter regions. The expression control sequences may be eukaryotic promoter systems in vectors capable of transforming or transfecting eukaryotic host cells. Once the vector has been incorporated into the appropriate host, the host is maintained under conditions suitable for high level expression of the nucleotide sequences, and for the collection and purification of the crossreacting antibodies.

A nucleic acid encoding the present antibody or antigen-binding portion thereof may be introduced into an expression vector that can be expressed in a suitable expression system, followed by isolation or purification of the expressed antibody or antigen-binding portion thereof. Optionally, a nucleic acid encoding the present antibody or antigen-binding portion thereof can be translated in a cell-free translation system, e.g., see U.S. Pat. No. 4,816,567.

The present disclosure also provides for cells comprising the nucleic acids described herein. The cells may be a hybridoma or transfectant. Examples of the cell types are discussed above.

Various techniques, such as production of chimeric or humanized antibodies, may involve procedures of antibody cloning and construction. The antigen-binding V_{L} (variable light chain) and V_{H} (variable heavy chain) sequences for an antibody of interest may be obtained by a variety of molecular cloning procedures, such as (but not limited to) RT-PCR, 5′-RACE, and cDNA library screening. The V_{L} and V_{H} genes of an antibody from a cell that expresses a murine antibody can be cloned by PCR amplification and sequenced. To confirm their authenticity, the cloned V_{L} and V_{H} genes can be expressed in cell culture as a chimeric antibody, for example (but not by way of limitation) as described by Orlandi et al. (Proc. Natl. Acad. Sci. USA, 86: 3833 (1989)). Based on the V_{L} and V_{H} gene sequences, a humanized antibody can then be designed and constructed as described by (for example but not by way of limitation) Leung et al. (Mol. Immunol., 32: 1413 (1995)).

As mentioned above, mAbs and mAb-derived compounds can be derivatized or linked to, e.g., conjugated to, the cardiac-targeting moieties disclosed herein. For example, an antibody can be functionally linked, directly or indirectly, by covalent bonding or by noncovalent interactions to one or more other molecular entities

Particularly suitable (but non-limiting) moieties for conjugation to the cardiac-targeting moieties or for loading into the liposomes disclosed herein include cytotoxic agents (e.g., chemotherapeutic agents), prodrug converting enzymes, radionuclides such as (but not limited to) radioactive isotopes or compounds, an immunomodulator, an anti-angiogenic agent, a pro-apoptotic agent, a cytokine, a chemokine, a drug, a hormone, an siRNA, an enzyme, a growth factor, a prodrug, an oligonucleotide, a pro-apoptotic agent, an interference RNA, a photoactive therapeutic agent, a tyrosine kinase inhibitor, a Bruton kinase inhibitor, a sphingosine inhibitor, a cytotoxic agent, or toxins (these moieties being collectively referred to as therapeutic agents or drugs). Examples of useful classes of cytotoxic agents include (but are not limited to) DNA minor groove binders, DNA alkylating agents, and tubulin inhibitors. Exemplary cytotoxic agents include (but are not limited to) auristatins, camptothecins, duocarmycins, etoposides, maytansines and maytansinoids (e.g., DM1 and DM4), taxanes, benzodiazepines (e.g., pyrrolo[1,4]benzodiazepines (PBDs), indolinobenzodiazepines, and oxazolidinobenzodiazepines), and vinca alkaloids. Techniques for conjugating therapeutic agents to proteins, and in particular to antibodies, are well-known (e.g., see, Carter, PJ and Senter PD, “Antibody-Drug Conjugates for Cancer Therapy.” Cancer J., 14(3):154-169 (2008)).

Examples of diagnostic agents include, but are not limited to: radionuclide, a contrast agent, a fluorescent agent, a chemiluminescent agent, a bioluminescent agent, a paramagnetic ion, an enzyme, and a photoactive diagnostic agent.

Examples of radionuclides include, but are not limited to: ¹¹¹In, ¹¹¹At, ¹⁷⁷Lu, ²¹¹Bi, ²¹²Bi, ²¹³Bi, ²¹¹At, ⁶²Cu, ⁶⁷Cu, ⁹⁰Y ¹²⁵I, ¹³¹I, ¹³³I, ³²P, ³³P, ⁴⁷Sc, ¹¹¹Ag, ⁶⁷Ga, ¹⁵³Sm, ¹⁶¹Tb, ¹⁵²Dy, ¹⁶⁶Dy, ¹⁶¹Ho, ¹⁶⁶Ho, ¹⁸⁶Re, ¹⁸⁸Re, ¹⁸⁹Re, ²¹¹Pb, ²¹²Pb, ²²³Ra, ²²⁵Ac, ²²⁷Th, ⁷⁷As, ⁸⁹Sr, ⁹⁹Mo, ¹⁰⁵Rh, ¹⁴⁹Pm, ¹⁶⁹Er, ¹⁹⁴Ir, ⁵⁸Co, ^{80m}Br, ^{99m}Tc, ^{103m}Rh, ¹⁰⁹Pt, ¹¹⁹Sb, ^{189m}Os, ¹⁹²Ir, ²¹⁹Rn, ²¹⁵Po, ²²¹Fr, ²⁵⁵Fm, ¹¹C, ¹³N, ¹⁵O, ⁷⁵Br, ¹⁹⁸Au, ¹⁹⁹Au, ²²⁴Ac, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ²²⁷Th, ¹²⁵Te, ¹⁶⁵Tm, ¹⁶⁷Tm, ¹⁶⁸Tm, ¹⁹⁷Pt, ¹⁰⁹Pd, ¹⁴²Pr, ¹⁴³Pr, ¹⁶¹Tb, ⁵⁷Co, ⁵⁸Co, ⁵¹Cr, ⁵⁹Fe, ⁷⁵Se, ²⁰¹Tl, ⁷⁶Br, ¹⁶⁹Yb, ¹¹⁰In, ¹⁸F, ⁵²Fe, ⁶⁴Cu, ⁶⁸Ga, ⁸⁶Y, ⁸⁹Zr, ^{94m}Tc, ⁹⁴Tc, ¹²⁰I, ¹²³I, ¹²⁴I, ¹⁵⁴⁻¹⁵⁸Gd, ³²F, ¹¹C, ¹³N, ⁵¹Mn, ^{52m}Mn, ⁵⁵Co, ⁷²As, ⁷⁵Br, ⁷⁶Br, ^{82m}Rb, ⁸³Sr, or other gamma-, beta-, or positron-emitters.

Examples of paramagnetic ions include, but are not limited to: chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), and erbium (III).

Examples of fluorescent labeling diagnostic agents include, but are not limited to: fluorescein isothiocyanate, rhodamine, phycoerytherin, phycocyanin, allophycocyanin, o-phthaldehyde, and fluorescamine; or a chemiluminescent labeling compound selected from the group comprising luminol, isoluminol, an aromatic acridinium ester, an imidazole, an acridinium salt, and an oxalate ester; or a bioluminescent compound selected from the group comprising luciferin, luciferase, and aequorin.

Therapeutic and/or diagnostic agents may include, without limitation, immunomodulators, cytokines (and their inhibitors), chemokines (and their inhibitors), tyrosine kinase inhibitors, growth factors, hormones and certain enzymes (i.e., those that do not induce local necrosis), or their inhibitors.

Examples of therapeutic agents that can be used as cargo molecules in the liposomes and compositions of the present disclosure include, but are not limited to, drugs to treat myocardial infarction, myocardial ischemia, reperfusion injury, congestive heart failure (CHF), cardiomyopathies, coronary artery disease (CAD), atrial fibrillation, inflammation, atherosclerosis, unstable angina, arrhythmias, valve diseases, congenital and inherited heart conditions, and heart infections.

Examples of types of drugs for treating heart failure that may be used in the compositions of the present disclosure include but are not limited to ACE inhibitors, such as benazepril, captopril, cilazapril, delapril, enalapril, fosinopril, lisinopril, perindopril, quinapril, ramipril, spirapril, trandolapril, and zofenopril; angiotensin-2 receptor blockers (ARBs) such as azilsartan, candesartan, eprosartan, fimasartan, forasartan, irbesartan, losartan, olmasartan, pratosartan, saparasartan, saralasin, tasosartan, telmisartan, and valsartan; beta blockers such as atenolol, acebutolol, bisoprolol, bucindolol, carvedilol (Coreg), celiprolol, esmolol, labetolol, metoprolol, nadolol, nebivolol, pindolol, propranolol, sotalol, and timolol; calcium channel blockers such as amlodipine, diltiazem (Cardizem), felodipine, isradipine, nicardipine, nifedipine (Procardia), nisoldipine (Sular), verapamil (Calan SR); mineralocorticoid receptor antagonists such as canrenone, eplerenone, finerenone, and spironolactone; ivabradine; sacubitril valsartan; hydralazine with nitrate; and digoxin.

Examples of other therapeutic agents include, but are not limited to: 5-fluorouracil, aplidin, azaribine, anastrozole, anthracyclines, bendamustine, bleomycin, bortezomib, bryostatin-1, busulfan, calicheamycin, camptothecin, carboplatin, 10-hydroxycamptothecin, carmustine, celecoxib, chlorambucil, cisplatinum, Cox-2 inhibitors, CPT-11 SN-38, carboplatin, cladribine, camptothecans, cyclophosphamide, cytarabine, dacarbazine, docetaxel, dactinomycin, daunorubicin, doxorubicin, 2-pyrrolinodoxorubicine (2P-DOX), pro-2P-DOX, cyano-morpholino doxorubicin, doxorubicin glucuronide, epirubicin glucuronide, estramustine, epipodophyllotoxin, estrogen receptor binding agents, etoposide (VP16), etoposide glucuronide, etoposide phosphate, floxuridine (FUdR), 3′,5′-O-dioleoyl-FudR (FUdR-dO), fludarabine, flutamide, farnesyl-protein transferase inhibitors, gemcitabine, hydroxyurea, idarubicin, ifosfamide, L-asparaginase, lenolidamide, leucovorin, lomustine, mechlorethamine, melphalan, mercaptopurine, 6-mercaptopurine, methotrexate, mitoxantrone, mithramycin, mitomycin, mitotane, navelbine, nitrosourea, plicomycin, procarbazine, paclitaxel, pentostatin, PSI-341, raloxifene, semustine, streptozocin, tamoxifen, paclitaxel, temazolomide, transplatinum, thalidomide, thioguanine, thiotepa, teniposide, topotecan, uracil mustard, vinorelbine, vinblastine, vincristine, a vinca alkaloid, a tyrophostin, canertinib, dasatinib, erlotinib, gefitinib, imatinib, lapatinib, leflunomide, nilotinib, pazopanib, semaxinib, sorafenib, sunitinib, sutent, vatalanib, PCI-32765 (ibrutinib), PCI-45292, GDC-0834, LFM-A13, and RN486.

Examples of toxins include, but are not limited to, ricin, abrin, alpha toxin, saporin, ribonuclease (RNase; e.g., onconase), DNase I, Staphylococcal enterotoxin-A, pokeweed antiviral protein, gelonin, diphtheria toxin, Pseudomonas exotoxin, and Pseudomonas endotoxin.

Immunomodulators include, but are not limited to, cytokines, stem cell growth factors, lymphotoxins, hematopoietic factors, colony stimulating factors (CSF), interferons (IFN), erythropoietins, thrombopoietins, and combinations thereof. Specifically useful are lymphotoxins such as (but not limited to) tumor necrosis factor (TNF); hematopoietic factors such as (but not limited to) interleukin (IL); colony stimulating factors such as (but not limited to) granulocyte-colony stimulating factor (G-CSF) or granulocyte macrophage-colony stimulating factor (GM-CSF); interferons such as (but not limited to) interferons-alpha, -beta, -lambda, or -gamma; and stem cell growth factors such as (but not limited to) that designated “S1 factor.” Included among the cytokines are growth hormones such as (but not limited to): human growth hormone, N-methionyl human growth hormone, and bovine growth hormone; parathyroid hormone; thyroxine; insulin; proinsulin; relaxin; prorelaxin; glycoprotein hormones such as (but not limited to) follicle stimulating hormone (FSH), thyroid stimulating hormone (TSH), and luteinizing hormone (LH); hepatic growth factor; prostaglandin; fibroblast growth factor; prolactin; placental lactogen; OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as (but not limited to)NGF-beta; platelet-growth factor; transforming growth factors (TGFs) such as (but not limited to) TGF-alpha and TGF-beta; insulin-like growth factor-I and —II; erythropoietin (EPO); osteoinductive factors; interferons such as (but not limited to) interferon-alpha, -beta, -lambda, and -gamma; colony stimulating factors (CSFs) such as (but not limited to) macrophage-CSF (M-CSF); interleukins (ILs) such as (but not limited to) IL-1, IL-1 alpha, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25; leukemia inhibitory factor (LIF); kit-ligand or FLT-3 ligand; angiostatin; thrombospondin; endostatin; tumor necrosis factor; and lymphotoxin. Chemokines of use include (but are not limited to): RANTES, MCAF, MIP1-alpha, MIP1-Beta, and IP-10.

In certain non-limiting embodiments, therapeutic radionuclides have a decay-energy in the range of 20 to 6,000 keV, such as (but not limited to) in the ranges of: 60 to 200 keV for an Auger emitter; 100-2,500 keV for a beta emitter; and 4,000-6,000 keV for an alpha emitter. Maximum decay energies of useful beta-particle-emitting nuclides may be, but are not limited to, 20-5,000 keV; 100-4,000 keV; or 500-2,500 keV. Also included are radionuclides that substantially decay with Auger-emitting particles, such as (but not limited to): Co-58, Ga-67, Br-80m, Tc-99m, Rh-103m, Pt-109, In-111, Sb-119, 1-125, Ho-161, Os-189m, and Ir-192. Decay energies of useful beta-particle-emitting nuclides may be (for example but not by way of limitation): <1,000 keV, <100 keV, or <70 keV Also included are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227, and Fm-255. Decay energies of useful alpha-particle-emitting radionuclides include (but are not limited to): 2,000-10,000 keV; 3,000-8,000 keV; or 4,000-7,000 keV.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, degree of immunoprotection desired, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) to form a pharmacological composition. Pharmaceutically acceptable carriers can contain a physiologically acceptable compound that acts to, for example but not by way of limitation) stabilize or increase or decrease the absorption or clearance rates of the pharmaceutical compositions. Physiologically acceptable compounds can include, for example but not by way of limitation: carbohydrates, such as glucose, sucrose, or dextrans; antioxidants, such as ascorbic acid or glutathione; chelating agents; low molecular weight proteins; detergents; liposomal carriers; excipients; or other stabilizers and/or buffers. Other physiologically acceptable compounds include (but are not limited to) wetting agents, emulsifying agents, dispersing agents, or preservatives.

In one aspect, the pharmaceutical formulations comprising compositions or nucleic acids, antibodies or fragments thereof are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the polypeptides or nucleic acids have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028, and as described elsewhere herein.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

The composition is formulated to contain an effective amount of the presently disclosed active agent, wherein the amount depends on the animal to be treated and the condition to be treated. In certain non-limiting embodiments, the active agent is administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 g to about 1.5 mg, from about 10 g to about 1 mg protein, from about 30 g to about 500 g, from about 40 g to about 300 g, from about 0.1 g to about 200 mg, from about 0.1 g to about 5 g, from about 5 g to about 10 g, from about 10 g to about 25 g, from about 25 g to about 50 g, from about 50 g to about 100 g, from about 100 g to about 500 g, from about 500 g to about 1 mg, or from about 1 mg to about 2 mg. The specific dose level for any particular subject depends upon a variety of factors, including (but not limited to) the activity of the specific peptide, the age, body weight, general health, sex, diet, time of administration, route of administration, and rate of excretion, the drug combination, and the severity of the particular disease undergoing therapy.

The dosage of an administered active agent for humans will vary depending upon factors such as (but not limited to) the patient's age, weight, height, sex, general medical condition, and previous medical history. In certain non-limiting embodiments, the recipient is provided with a dosage of the active agent that is in the range of from about 1 mg to about 1000 mg as a single infusion or single or multiple injections, although a lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Non-limiting examples of dosages of the active agent that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In some non-limiting embodiments, the effective amount of a cardiac-targeting moiety of the present disclosure is in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 M, about 3 μM, about 4 μM, about 5 μM, about 6 μM, about 7 μM, about 8 M, about 9 μM, about 10 μM, about 15 M, about 20 μM, about 25 μM, about 30 μM, about 35 μM, about 40 μM, about M, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

In some non-limiting methods, the patient is administered the active agent each one, two, three, or four weeks, for example. The dosage depends on the frequency of administration, condition of the patient, response to prior treatment (if any), whether the treatment is prophylactic or therapeutic, and whether the disorder is acute or chronic, among other factors.

Administration can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

In certain non-limiting embodiments, pharmaceutical compositions are sterile, substantially isotonic, and manufactured under GMP conditions. Pharmaceutical compositions can be provided in unit dosage form (i.e., the dosage for a single administration). Pharmaceutical compositions can be formulated using one or more physiologically acceptable carriers, diluents, excipients, or auxiliaries. The formulation depends on the route of administration chosen. For injection, the active agent can be formulated in aqueous solutions, such as (but not limited to) in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, the active agent can be in lyophilized form for constitution with a suitable vehicle, e.g., sterile pyrogen-free water, before use. The concentration of the active agent in a liquid formulation can be for example (but not by way of limitation) 0.01-10 mg/ml, such as 1.0 mg/ml.

### EXPERIMENTAL

Having generally described embodiments drawn to head-linker-anchor targeting vectors, cargo-loaded cardiac-targeted liposomes, and cardiac-targeted drug conjugates, and methods of their use, a fuller understanding of the presently disclosed technology can be obtained by reference to certain specific examples which are provided below for purposes of illustration only and are not intended to be limiting of the inventive concepts described herein.

### Example 1

In one non-limiting embodiment, a conjugate OM-PEG-distearoylphosphatidylethanolamine (“OM-PEG-DSPE” or “DSPE-PEG-OM”) was synthesized. An OM precursor, compound 4 was first synthesized via Scheme 1. Then, compound 4 was used to synthesize compound 5 (OM-PEG-DSPE) via Scheme 2 using PEG₂₀₀₀. The OM-PEG-DSPE was then used to make the cardiac-targeted liposomes that were used in the experiments described below (Example 2).

### Synthesis of Compound 4 (Scheme 1)

To synthesize compound 4, 1-(bromomethyl)-2-fluoro-3-nitrobenzene was reacted with mono BOC-protected piperazine in presence of diisopropylethylamine (DIPEA). After water workup tert-butyl 4-(2-fluoro-3-nitrobenzyl)piperazine-1-carboxylate (compound 1) was obtained, which was reduced by hydrogen gas using catalytic Pd/C to obtain tert-butyl 4-(3-amino-2-fluorobenzyl)piperazine-1-carboxylate (2). Urea analog 3 was prepared by reacting O-benzoyl-N-(6-methylpyridin-3-yl)hydroxylamine with compound 2 in presence of DIPEA. Upon completion of the reaction, the mixture was evaporated, and residue was purified by silica column using ethyl acetate/hexane (50:50 v/v). Compound 3 was treated with trifluoroacetic acid (TFA), and residue was purified by column to obtain 1-(2-fluoro-3-(piperazin-1-ylmethyl)phenyl)-3-(6-methylpyridin-3-yl)urea (compound 4) as TFA salt. The compound was basified with NaOH solution and extracted with ethyl acetate, dried with sodium sulfate, and evaporated to collect compound 4 in base form.

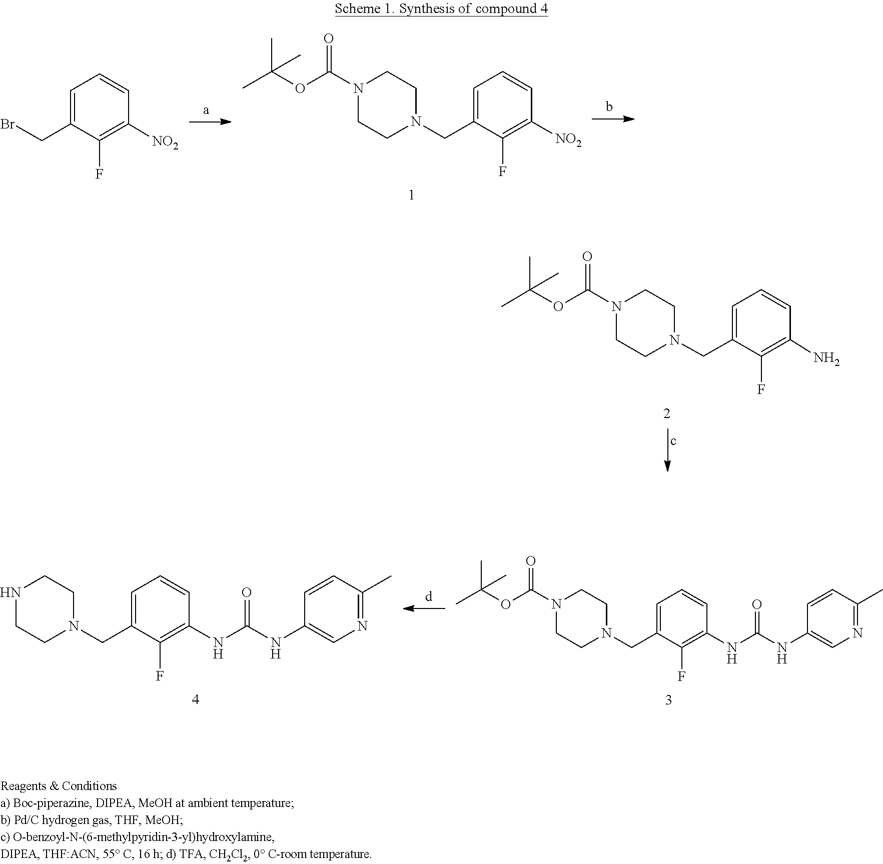 [SMILES: O=[N+]([O-])c1cccc(CBr)c1F ; CC(C)(C)OC(=O)N1CCN(Cc2cccc([N+](=O)[O-])c2F)CC1 ; CC(C)(C)OC(=O)N1CCN(Cc2cccc(N)c2F)CC1 ; Cc1ccc(NC(=O)Nc2cccc(CN3CCNCC3)c2F)cn1 ; Cc1ccc(NC(=O)Nc2cccc(CN3CCN(C(=O)OC(C)(C)C)CC3)c2F)cn1]

### Synthesis of compound 5 (OM-PEG-DSPE, Scheme 2)

Compound 4 was reacted with distearoylphosphatidylethanolamine-PEG₂₀₀₀-N-hydroxysuccinimide ester (DSPE-PEG₂₀₀₀-NHS ester) (BroadPharm®) in presence of dimethylaminopyridine (DMAP) in dry dimethylformamide (DMF). After completion of the reaction, DMF was evaporated, and residue was purified on neutral alumina using methanol/chloroform (5:95 v/v). The isolated compound was characterized by ¹H-NMR and ¹³C-NMR (see Supporting Information below).

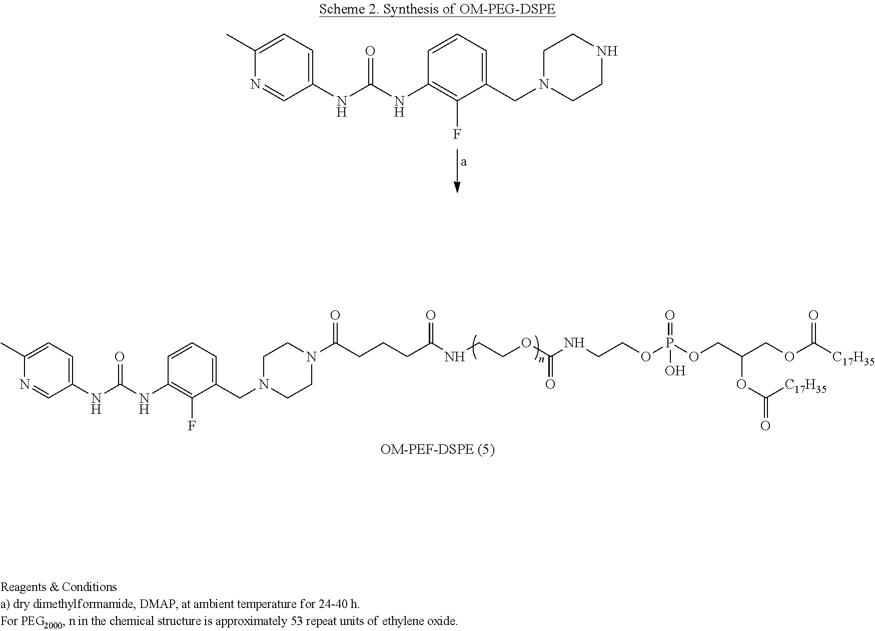

### Supporting Information

### Materials

All commercial chemicals were used as obtained and all solvents were purified by the standard procedures prior to use. Flash column chromatography was performed with E Merck silica gel (230-400 mesh). NMR spectra were measured against the peak of tetramethylsilane by Varain Unity Inova 400 NMR (400 MHz) spectrometers. All tested compounds were evaluated on the Agilent HPLC systems usingACE-C18 column (250×4.6 mm) was used as the stationary phase. HPLC conditions include a flow rate of 1.0 mL/min using water and acetonitrile as solvents and a detection wavelength of 254 nm and determined to be >95% pure.

### Tert-butyl 4-(2-fluoro-3-nitrobenzyl)piperazine-1-carboxylate (1)

To a solution of 1-(bromomethyl)-2-fluoro-3-nitrobenzene in methanol was added tert-butyl piperazine-1-carboxylate (1 equivalent) and DIPEA (2 equivalents) stirred for 5 h at ambient temperature. After completion of reaction evaporated the organic layer, residue was subjected to column chromatography. 85% yield. ¹H NMR (CDCl₃, 400 MHz) δ: 7.06 (t, J=7.71, 1H), 6.89 (d, J=7.08, 1H), 6.73 (d, J=7.89, 1H), 3.65 (s, 2H), 3.53 (s, 4H), 2.53 (s, 4H), 1.35 (s, 9H).

### Tert-butyl 4-(3-amino-2-fluorobenzyl)piperazine-1-carboxylate (2)

To a solution of tert-butyl 4-(2-fluoro-3-nitrobenzyl)piperazine-1-carboxylate in tetrahydrofuran/methanol mixture was added 5% Pd/C and bubbled hydrogen gas (balloon) for 3-5 h at room temperature. After TLC confirmation of reaction completion, filtered through celite and evaporated to get amino-derivative (2) which was used further without purification. 92% yield. ¹H NMR (CDCl₃, 400 MHz) δ: 7.06 (t, J=7.71, 1H), 7.08 (d, J=7.08, 1H), 7.86 (d, J=7.89, 1H), 4.11 (s, 2H), 3.65 (s, 2H), 3.53 (broad peak, 4H), 2.53 (broad peak, 4H), 1.50 (s, 9H).

### Tert-butyl 4-(2-fluoro-3-(3-(6-methylpyridin-3-yl)ureido)benzyl)piperazine-1-carboxylate (3)

To a solution of tert-butyl 4-(3-amino-2-fluorobenzyl)piperazine-1-carboxylate (1 equivalents) 2 in THF (5 volumes) was added O-benzoyl-N-(6-methylpyridin-3-yl)hydroxylamine followed by acetonitrile (5 volumes) to the above slurry DIPEA was added drop wise and heated to 55-60° C. for 16 h. After completion of reaction solvent was evaporated and residue was purified by flash column chromatography to get desired compound 3 in 70% yield. ¹H NMR (CDCl₃, 400 MHz) δ: 8.34 (m, 2H), 8.17 (t, J=7.30, 1H), 8.05 (m, 2H), 7.12 (d, J=8.49, 1H), 7.06 (t, J=7.89, 1H), 6.94 (t, J=6.48, 1H), 3.54 (s, 2H), 3.47 (broad peak, 4H), 2.52 (s, 3H), 2.42 (broad peak, 4H), 1.50 (s, 9H).

### 1-(2-fluoro-3-(piperazin-1-ylmethyl)phenyl)-3-(6-methylpyridin-3-yl)urea (4)

To a solution of compound 3 in dichloromethane added 2 volumes of TFA and stirred for 2 h at room temperature. After completion of deprotection evaporated the reaction mixture and to the residue ether was added and obtained solid was collected to get desired product in 90% yield. ¹H NMR (DMSO-d₆, 400 MHz) δ: 10.29 (s, 1H), 9.33 (s, 1H), 8.99 (d, J=2.25, 2H), 8.25 (dd, J=3.69 1H), 8.04 (m, 1H), 7.79 (d, J=8.78, 1H), 7.21 (m, 2H), 4.09 (s, 2H), 3.26 (s, 2H), 3.04 (s, 4H), 2.63 (s, 3H).

### Synthesis of DSPE-Linked Omacamtiv Mecabril (OM-PEG-DSPE)

To a solution of DSPE-PEG₂₀₀₀-NHS ester (1 equivalent) in dry dimethylformamide (2 volumes) at 0-5° C. was added 1-(2-fluoro-3-(piperazin-1-ylmethyl)phenyl)-3-(6-methylpyridin-3-yl)urea (1.01 equivalents) under argon atmosphere. After mixing, dimethylaminopyridine (1.0 equivalents) was added and the mixture was stirred for 24-40 h. Reaction progress was monitored by thin-layer chromatography using 9.5:0.5 ethyl acetate/methanol developing solvent. After 40 h, the solvent was evaporated on a rotary evaporator and the dry residue was purified on neutral activated alumina using acetonitrile-water system. The collected fractions were evaporated to dryness to get the desired product.

### Example 2

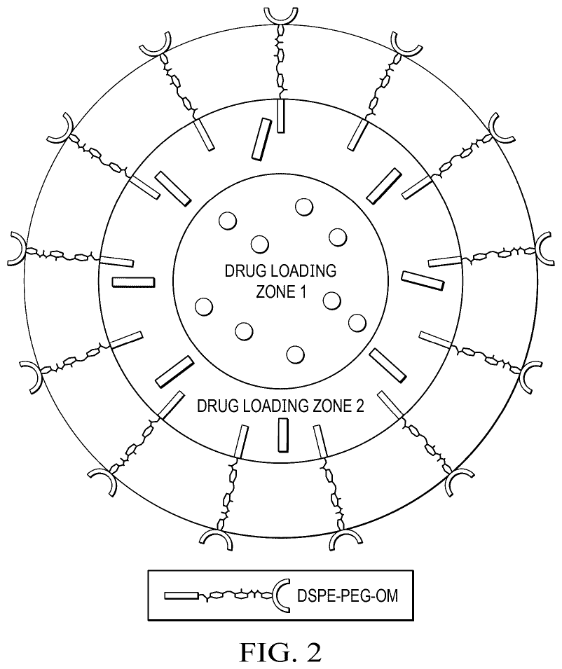

The OM-PEG-DSPE (compound 5) was incorporated into a liposome composition as a targeting vector. A graphic representation of the loaded liposome is shown in FIG. 2. The OM end of the targeting vector extends from the surface of the liposome and the DSPE end is embedded in the liposome. Preferential accumulation of the resulting OM-liposomes in infarcted heart is demonstrated by molecular imaging in live mice. The results show that OM-liposomes can be employed to deliver cardio-protective therapeutics in acute myocardial infarction (AMI) to reduce myocardial injury and potentiate post-ischemia tissue recovery.

### Preparation of OM-PEG-DSPE-liposomes (OM-liposomes)

Liposomes of composition dipalmitoylphosphatidylcholine (DPPC), cholesterol, and OM-PEG-DSPE (6:3:1 M ratio) were made by extrusion followed by post-insertion of OM-PEG-DSPE. Briefly, DPPC and cholesterol were dissolved in the chloroform-methanol mixture (3:1). After filtration, through a 0.2 μm nylon filter, the solvent was removed on an R-210 rotary evaporator (Buchi Corporation, New Castle, DE, USA) to obtain a thin film of lipid phase deposited inside a round bottom flask. The film was hydrated with ammonium sulfate solution (300 mM, pH 4.0). The lipid suspension was subjected to 10 freeze-thaw cycles and then sequentially passed five times each through polycarbonate membranes with pore size of 1, 0.8, 0.6, 0.4, 0.2, and 0.1 μm. After extrusion, the outer bilayers of liposomes were inserted with OM-PEG-DSPE. To do this, the liposome preparation (˜5 mg/mL phospholipid) was incubated with a micellar suspension of OM-PEG-DSPE (10 mole percent with respect to the total phospholipid amount). The incubation temperature was set at 40° C., a value close to the transition temperature Tₘ of DPPC. The reaction was allowed to proceed for an hour under slow and continuous stirring. Afterwards, the temperature was allowed to gradually equilibrate to room temperature. The resultant post-inserted liposomes were separated by centrifugation at 137,000×g and 4° C. for 1 h in Beckman Optima L-100 XP ultracentrifuge (Fullerton, CA, USA). The liposome pellet was suspended in water and re-centrifuged. After two wash-cycles, the pellet was re-suspended in water to phospholipid concentration of approximately 20 mg/mL. Phospholipid concentration was determined by Stewart colorimetric assay. The liposome size was assessed by dynamic light-scattering Zeta PALS instrument (Brookhaven Instruments Corporation, Holtsville, NY, USA).

To histologically document accumulation of OM-liposomes in the myocardial tissue, we also made OM-liposomes which contained Texas Red (TR) in the bilayer using the method described above, except the lipid phase was doped with TR dye. Approximately 333 ng of TR was incorporated in lipid bilayer for each mg of DPPC. Table 1 shows the characteristics of OM-liposomes and the Texas Red OM-liposomes (TR-OM-liposomes).

TABLE 1
Characteristics of liposomes

| Parameter | OM-liposomes | TR-OM-liposomes |
|---|---|---|
| Particle size (nm) | 112 | 148 |
| Polydispersity | 0.055 | 0.078 |
| Charge (mV) | ND | −46.8 |
| Phospholipid concn. | 30.63 | 22.33 |
| (mg/mL) | | |

Radiolabeling of OM-liposomes with ^{99m}Tc

Liposomes were labeled with ^{99m}Tc-BMEDA (N,N-bis(2-mercaptoethyl)-N′,N′-diethylethylenediamine, a lipophilic chelator which has been used for radiolabeling of liposomes. To prepare ^{99m}Tc-BMEDA by transchelation, ^{99m}Tc-glucoheptonate (GHA) was prepared by adding 10 mCi of Na^{99m}TcO₄ to a Sn²⁺-GHA kit vial (Hexakit, Inc, Oklahoma City, OK), and allowing 10 min incubation at ambient temperature. After confirming the formation of ^{99m}Tc-GHA complex by iTLC in acetone and saline, we added 5 μL of BMEDA and heated the mixture to 80° C. for 15 min. The mixture was cooled to ambient temperature and checked with paper chromatography (Whatman #1) in methanol, acetone, and saline. ^{99m}Tc-BMEDA travels with Rf of 0.65 in methanol, 0 in acetone, and 0.65 in saline, whereas ^{99m}Tc-GHA travels with Rf 0.65, 0.55, and 0.55, respectively. The resultant ^{99m}Tc-BMEDA complex was added to OM-liposomes followed by incubation 1 h at 37° C. Radiolabeled ^{99m}Tc-OM-liposomes were separated from free radioactivity by gel-exclusion chromatography on a PD10 column using PBS as the eluting buffer.

### Mouse Model of MI

All animal work was approved by the Institutional Animal Care and Use Committee of the University of Oklahoma Health Sciences Center. We used a mouse model that creates an infarct mimicking the coronary artery blockage produced by coronary artery disease in humans as described previously (Awasthi V, Gali H, Hedrick A F, Da H, Eeda V, Jain D. Positron Emission Tomography (PET) with ¹⁸F-FGA for Diagnosis of Myocardial Infarction in a Coronary Artery Ligation Model. Mol Imaging. 2022; 2022:9147379). CD1 mice (25-27 g, female, 6-8 weeks) were acclimatized for at least 5 days under a standardized light/dark cycle and allowed ad libitum access to rodent chow and tap water. Mice were anesthetized and endotracheally intubated for ventilation with 1.5% isoflurane-02 stream. A 1-5 cm incision was made in the 4^{th}-5^{th} intercostal space. The pericardium was opened to locate and permanently occlude the left anterior descending (LAD) artery using 8-0 nylon suture. After closing the incision, the mice were kept on O₂-ventilator until spontaneous breathing resumed. Saline (0.2 mL i.p.) and buprenorphine (1 mg/kg s.c.) were administered for hydration and analgesia, respectively. Sham control group only went through chest opening surgery without ligation of coronary artery.

### Planar Nuclear Imaging and Terminal Biodistribution

Planar gamma camera scans of mice were performed with nanoSPECT (Bioscan, Inc., Washington, DC, USA) using multi-pinhole collimators (0.3 mm diameter). All mice were under isoflurane anesthesia during the imaging. Projection data were acquired after 3 h of ^{99m}Tc-OM-liposomes for energy 140 keV for ^{99m}Tc. The animals were intravenously injected with 0.25-1.0 mCi of ^{99m}Tc-OM-liposomes in 50-150 μL volume.

Post-imaging the mice were returned to the cages and housed for examination of ^{99m}Tc-liposome distribution in various organs. The mice were euthanized after 24 h of injection by isoflurane overdose followed by cervical dislocation. Various organs and blood were collected, washed thoroughly with saline, and the radioactivity associated with tissues was counted in an automated gamma counter. The radioactivity counts were corrected for radioactive decay (t_{1/2} 6 h), and the uptake of OM-liposomes was expressed as injected dose per gram (% ID/g). Total blood volume, bone, and muscle mass were estimated as 5.7%, 10%, and 40% of body weight, respectively.

### Histology

After imaging, the mice were euthanized and the heart was isolated and sectioned for staining with 2,3,5-triphenyltetrazolium chloride (TTC). Briefly, the heart was sliced into 1-2 mm sections, and the sections were stained with 1% solution of TTC. After 20 min of staining, the sections were fixed in 10% phosphate-buffered formalin.

### Myocardial Uptake of TR-OM-Liposomes

TR-OM-liposomes (0.05 mL) were injected intravenously in mice with or without MI. After 4 h, the mice were killed to excise heart for sectioning. Briefly, 5 μm sections were formalin-fixed and mounted on glass slides for fluorescence microscopy using Leica DM4000 B (Illinois, US).

### Results

### Cardiotropic Property of OM-Liposomes

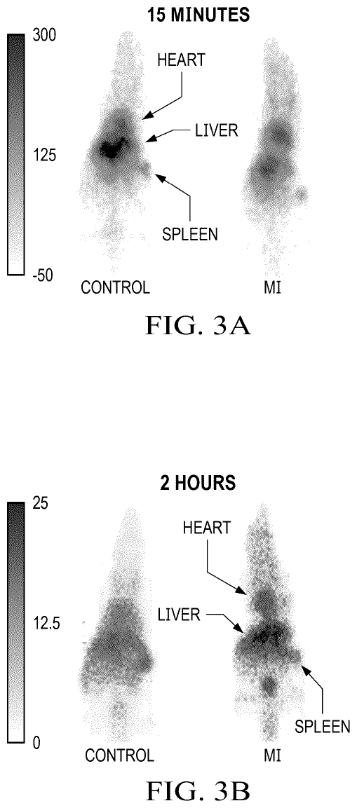

The in vivo cardiac targeting ability of OM-liposomes in mice was assessed by injecting 50 μL of ^{99m}Tc-labeled OM-liposomes (250 μCi). Planar gamma camera images were recorded at 15 min showed mostly blood pool of ^{99m}Tc-OM-liposomes in circulation (FIG. 3a). Images acquired after 2 h of injection showed higher uptake ^{99m}Tc-OM-liposomes in infarcted heart as compared to that in sham heart (FIG. 3b). There were significant circulating levels of ^{99m}Tc-OM-liposomes, perhaps attributable to the presence of PEG₂₀₀₀ in the bilayer. As expected, a large portion of injected ^{99m}Tc-OM-liposomes accumulated in liver and spleen.

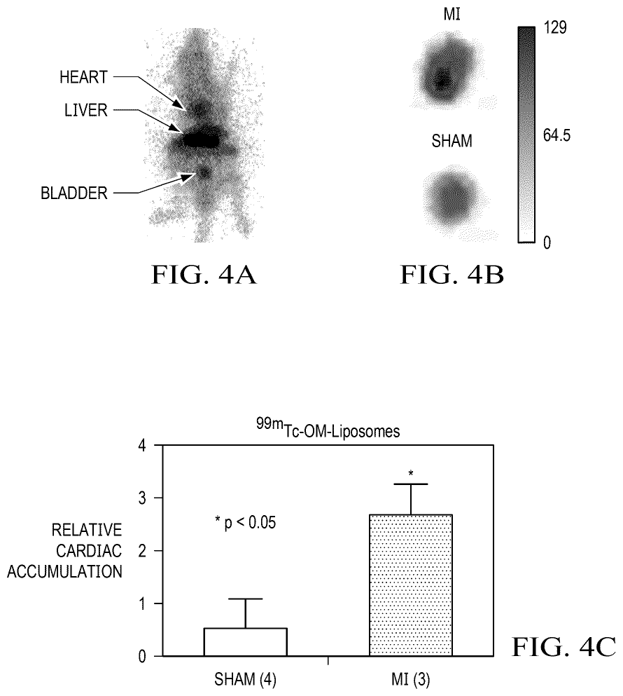

To confirm the high accumulation in the infarcted heart model, animals were sacrificed after 24 h of injection, hearts were collected from sham mice and MI mice and ex vivo images were acquired after washing with saline. The ex vivo images clearly showed larger uptake of ^{99m}Tc-OM-liposomes in MI hearts as compared to the sham control hearts (FIG. 4b). The excised hearts were also counted in a well counter and the data of heart-associated ^{99m}Tc-OM-liposomes are shown in FIG. 4c. The radioactive counts showed significant difference in uptake of ^{99m}Tc-OM-liposomes between the MI and sham control hearts.

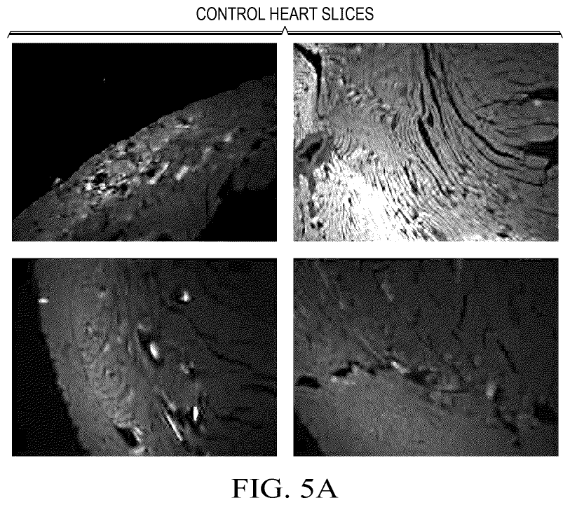

Finally, myocardial uptake of OM-liposomes in MI heart was monitored by fluorescent microscopy using Texas Red labeled OM-liposomes. The results in FIGS. 5A-5B show a clear distinction in uptake of OM-liposome between control hearts and MI hearts, respectively.

To document whole body biodistribution, we next examined 24 h uptake of ^{99m}Tc-OM-liposomes in various organs of control and MI mice. The radioactivity data are shown in Table 2.

TABLE 2
Biodistribution of ^{99m}Tc-OM-liposomes in mice¹

| Organ | Control (n = 4) | MI (n = 4) | p-value |
|---|---|---|---|
| Blood | 0.083 ± 0.062 | 0.140 ± 0.101 | 0.343 |
| Spleen | 3.953 ± 3.000 | 5.798 ± 2.250 | 0.486 |
| Liver | 1.745 ± 0.839 | 3.088 ± 1.230 | 0.314 |
| Kidney | 0.848 ± 0.652 | 1.525 ± 1.392 | 0.314 |
| Lung | 0.190 ± 0.123 | 0.368 ± 0.166 | 0.286 |
| Heart | 0.055 ± 0.035 | 0.160 ± 0.065 | 0.029* |
| Muscle | 0.035 ± 0.021 | 0.050 ± 0.037 | 0.543 |
| Bone | 0.065 ± 0.026 | 0.140 ± 0.109 | 0.571 |

¹= (% injected dose per gram tissue (*p < 0.05)

### Example 3

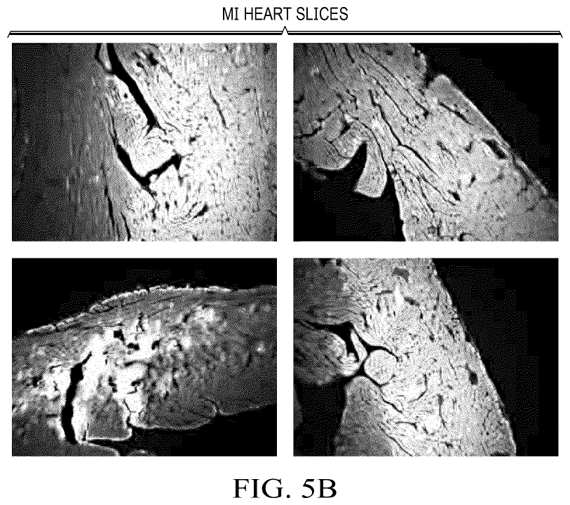

In another non-limiting embodiment of the present disclosure, the OM or OM-liposomes can be labeled with ¹⁸F radionuclide for PET imaging of myocardial infarction as an infarct-avid radiotracer. Unlike radiotracers for perfusion imaging, infarct-avid agents are not taken up by normal myocardium and only accumulate at the site of acute infarct. Therefore, infarct radiotracers produce images with less background and improved signal-to-noise ratio. Currently, no infarct-avid radiotracer is available for clinical PET imaging of infarct tissue. Preclinical studies demonstrated that ¹⁸F-OM detected surgical MI in mice with high target/non-target ratio (FIG. 6). Notable was the lack of background signal from accumulation in liver and lung tissue, which are major sources of artifacts in conventional imaging for myocardial perfusion.

### Example 4

### Direct Imaging of MI Using Tc-99m-Labeled OM and SPECT

In a non-limiting embodiment, specificity and sensitivity of detection of MI can be achieved by using ^{99m}Tc-omacamtiv, or a ^{99m}Tc-omacamtiv analog, with single photon emission tomography (SPECT). Results have demonstrated that ^{99m}Tc-omacamtiv detected surgical MI in mice with high target/non-target ratio. Notable was the lack of background signal from accumulation in liver and lung tissue, which are major sources of artifacts in conventional imaging for myocardial perfusion.

### Example 5

### Synthesis of Glucaric Acid (GA) Linked PEG-Biotin (GA-Biotin Probe)

DSPE-PEG₂₀₀₀-NHS Ester (BroadPharm®) was combined with omecamtiv (NH) analog prepared in our lab. To a solution of DSPE-PEG₂₀₀₀-NHS ester (1 equivalents) in dry DMF (2 volumes) at 0-5° C. was added 1-(2-fluoro-3-(piperazin-1-ylmethyl)phenyl)-3-(6-methylpyridin-3-yl)urea (1.01 equivalents) under argon atmosphere, followed by addition of diisopropylethylamine (1.2 equivalents) and stirred for 24-40h by monitoring TLC (9.5:0.5/EtOAc:MeOH). After 40 h of duration, solvents were taken to dryness and the residue was purified by neutral activated alumina (acetonitrile-water), the collected fractions were evaporated to dryness to get desired product.

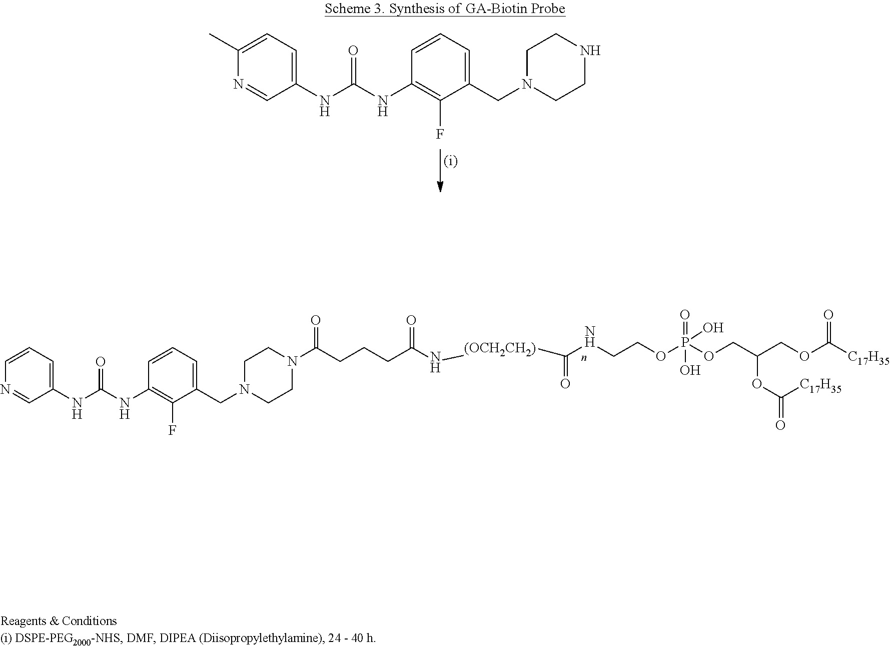

### Example 6

In certain embodiments, the present disclosure is directed to a method of imaging a heart in a subject in need of such treatment, comprising administering to the subject an ¹⁸F-labeled analog of OM (¹⁸F-OMA), and imaging the heart of the subject using positron emission tomographic (PET) imaging. For example, the subject may have previously experienced or is currently experiencing an episode of myocardial ischemia or myocardial infarction, or other cardiac condition in need of imaging or diagnosis.

In at least one embodiment, the ¹⁸F-labeled analog of OM has the structure:

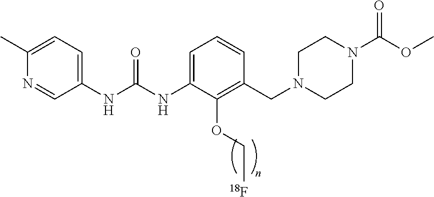

where n=2-10.

In another embodiment, ¹⁸F-OMA may replace the OM (head) moiety of the OM-linker-anchor targeting vectors discussed elsewhere herein.

### Example 7

In other embodiments, the OM analog or derivative that is used herein either as a cardiac treatment or as the head of the cardiac-myosin targeting vectors of the present disclosure is selected from the group consisting of:

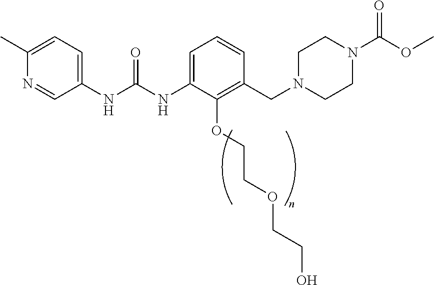

where n=1-2000, and

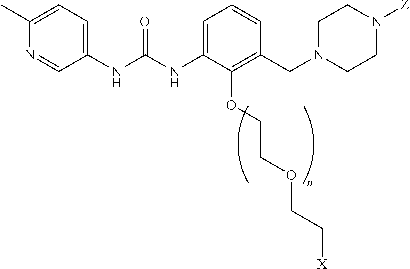

where
n=1-2000,
X═OH, OCH₃, F, ¹⁸F, and NH₂, and
Z═CH₃; acetyl; carbamoyl; —(CH₂)ₚCH₃, where p=1-20; or HO—(CH₂CH₂O)_{q}—H, where q=2-2,000.

### Example 8

As noted above, in certain embodiments, the present disclosure is directed to a method of imaging a heart in a subject in need of such treatment, comprising administering to the subject an ¹⁸F-labeled analog of OM (¹⁸F-OMA), and imaging the heart of the subject using positron emission tomographic (PET) imaging.

### Methods and Results

To enable radiolabeling with positron-emitting ¹⁸F radionuclide, first we synthesized a precursor where a tosyl leaving group was directly linked to the central benzene ring of omecamtiv. Tosyl moiety as a good leaving group that could be substituted by ¹⁸F in a radiolabeling procedure involving ¹⁸F-fluoride presented in a weak complex with Kryptofix_{2.2.2} as phase transfer catalyst in dry conditions. We followed an approach shown in Scheme 4. Reductive amination of piperazine carbamate with commercially available 2-hydroxy-3-nitrobenzaldehyde, followed by hydrogenation using H₂ gas, 10% Pd/C in 1:1 mixture of tetrahydrofuran and methanol at room temperature produced an aniline derivative 1. Urea derivative 2 was synthesized by reacting 1 with O-benzoyl-N-(6-methylpyridin-3-yl)hydroxylamine in presence of diisopropylethylamine (DIPEA) in 1:1 mixture of acetonitrile and tetrahydrofuran at 55-60° C. Precursor compound 3a (tosyl derivative) were prepared in dry DMF in presence of potassium carbonate by reacting 2 with tosyl chloride.

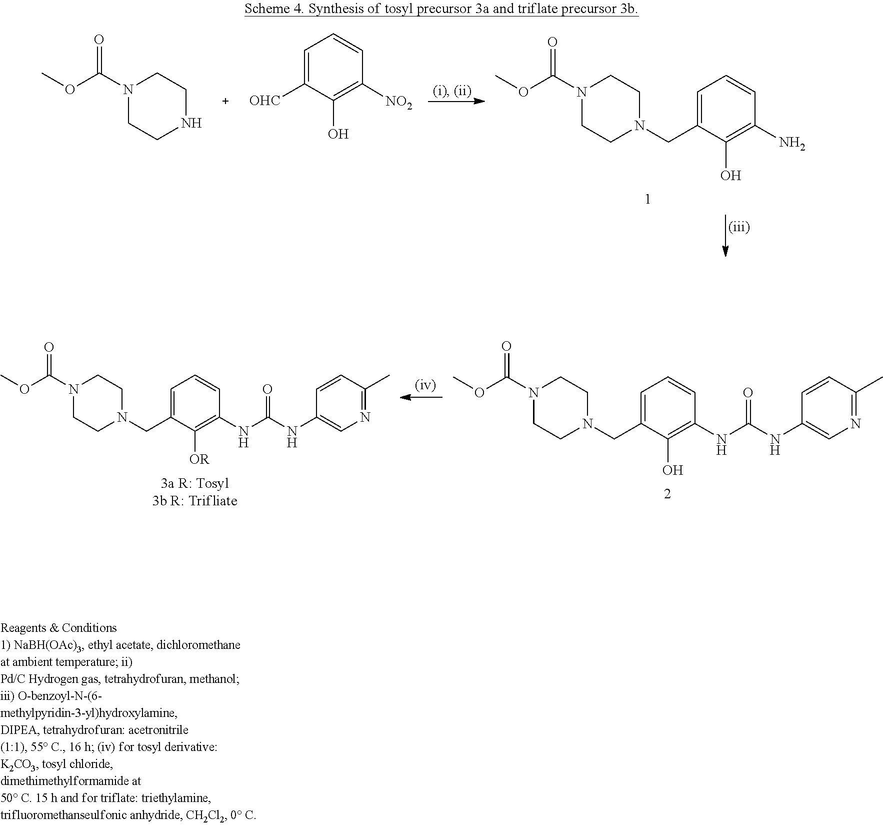

After obtaining pure 3a, we set ourselves for ¹⁸F radiolabeling using a conventional radiochemistry approach. However, our attempts to substitute tosyl group with ¹⁸F failed and we consistently observed p-¹⁸F-flurorotoluene as the ¹⁸F-labeled product accompanied by quantitative recovery of compound 2. These observations suggested that when tosyl leaving group is directly linked to the benzene ring, omecamtiv serves to activate tosyl and ¹⁸F fluoride prefers to attack the carbon at para to methyl group in tosyl moiety. The putative mechanism for this novel substitution is depicted in Scheme 5. To examine if triflate leaving group will be better than tosyl group, we also synthesized a triflate precursor 3b, but the radiolabeling yields were negligible for triflate precursor also.

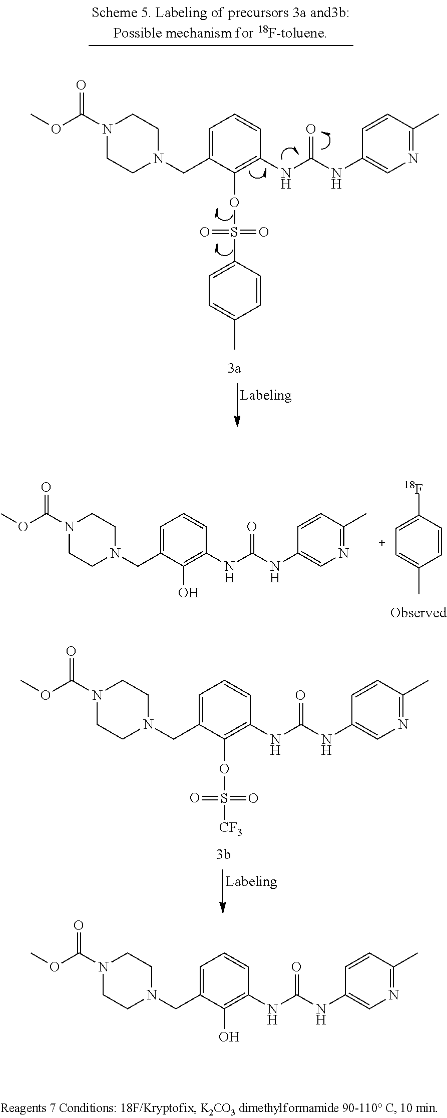

Next, we synthesized a precursor in which the tosyl leaving group was attached to the benzene ring via a 2-carbon linker (Scheme 6). O-alkylation of 2 was performed by 2-bromoethanol to obtain compound 4. O-tosylation of 4 to obtain compound 5 was achieved by using NaH as a base in dry tetrahydrofuran. Initial ¹⁸F radiolabeling experiments to radiolabel 5 were encouraging. Thus, we investigated various solvents and reaction temperatures to improve the yields. A 10 min reaction in dimethylformamide at 90° C. with Kryptofix_{2.2.2} presentation of ¹⁸F-fluroride provided maximum labeling yields. The organic solvent was evaporated by azeotropic evaporation using acetonitrile to obtain desired ¹⁸F-OM in crude form. A semipreparative C18 HPLC-based purification step was used to purify the crude preparation. An HPLC profile showed that the ¹⁸F-OM was pure. The average overall decay-corrected radiochemical yield was 45±8.5% (calculated from 5 independent labeling records), and the radiochemical purity of HPLC-purified ¹⁸F-OM was >95%.

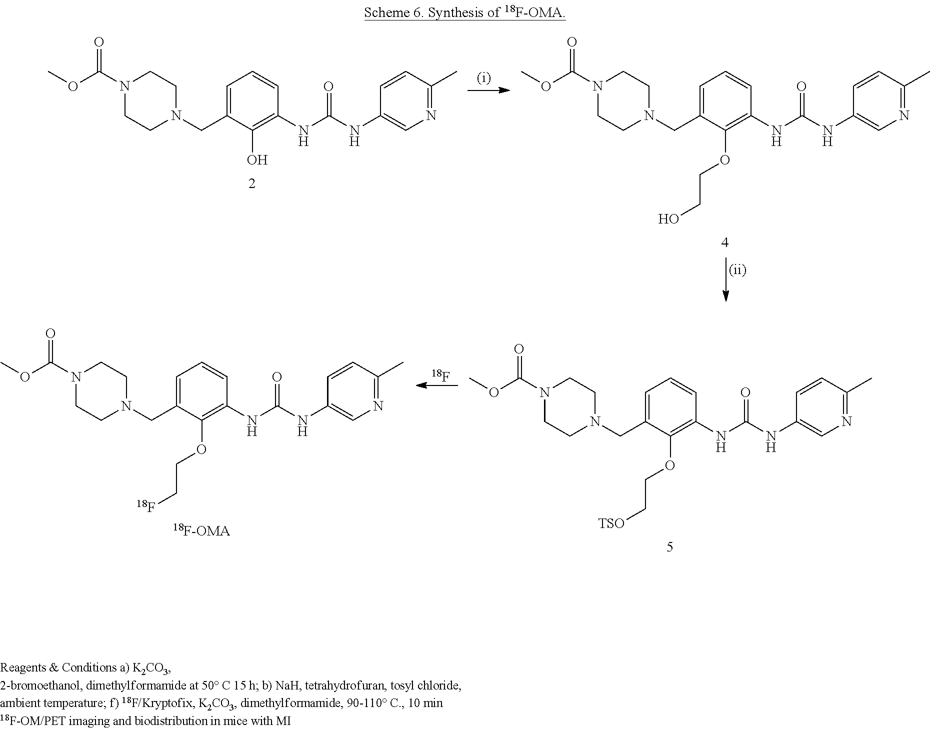

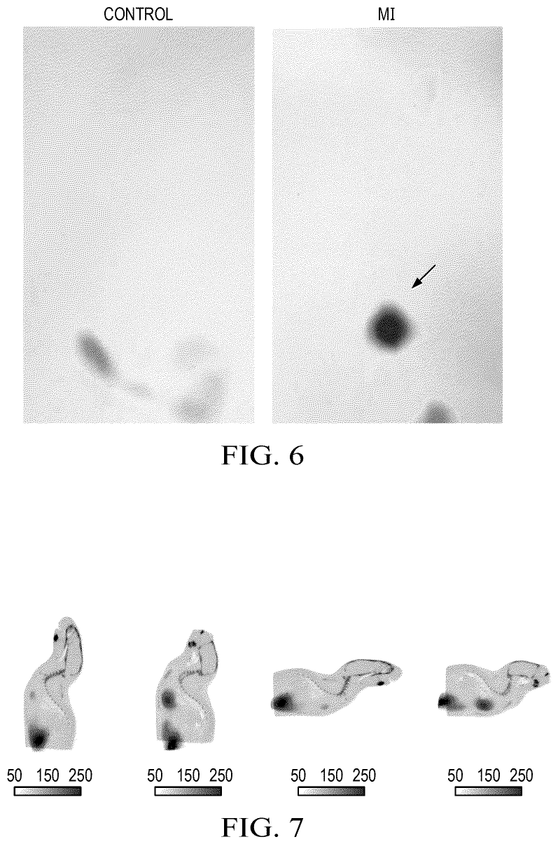

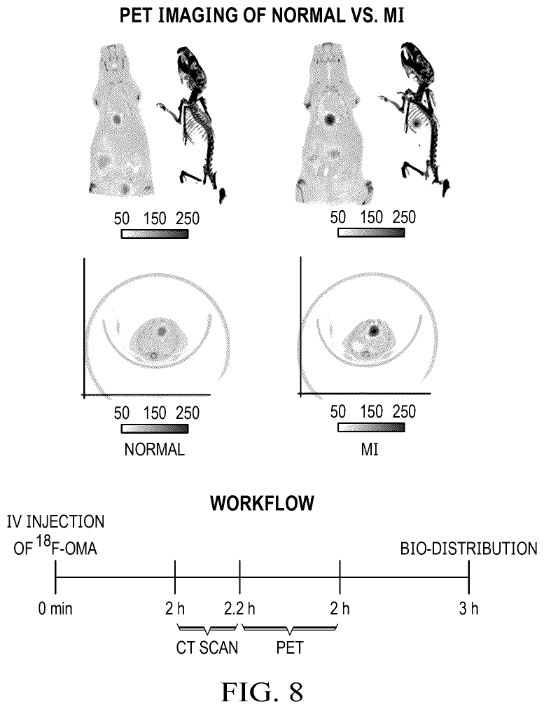

After successful labeling, we investigated ¹⁸F-OM for detection of MI in a mouse model of occluded anterior descending branch of the left coronary artery. After imaging, the mice were euthanized to collect various organs for counting of tissue-associated radioactivity. FIG. 7 shows the PET images acquired after x h of MI induction. Sham hearts accumulated significant ¹⁸F-OM, but the uptake in MI hearts was significantly more pronounced. Analysis of images for heart-associated radioactivity indicated that compared to normal hearts, MI hearts accumulated x times ¹⁸F-OM.

### Experimental

### Methyl 4-(2-hydroxy-3-nitrobenzyl)piperazine-1-carboxylate (1)

To a slurry of 2-hydroxy-3-nitrobenzaldehyde (1.0 equivalents) and methyl piperazine-1-carboxylate (1.2 equivalents) in a mixture of ethyl acetate (2 volumes) and dichloromethane (3 volumes) was added sodium triacetoxyborohydride (2.2 equivalents) as a solid over approximately 15 minutes. The reaction was stirred at room temperature for 5 hours. The reaction mixture was quenched with saturated. aq. sodium bicarbonate and then diluted with ethyl acetate. The layers were separated, and the aqueous layer was washed three times with ethyl acetate. The organic layers were combined and washed with brine, dried over anhydrous sodium sulfate, and concentrated under vacuum. Purification by flash chromatography provided the title compound (77%) as a yellow solid. ¹H NMR (CDCl₃, 400 MHz) δ: 8.00 (d, J=8.46 Hz, 1H), 7.55 d (d, J=7.38 Hz, 1H), 6.95 (t, J=7.87 Hz, 1H), 3.79 (s, 2H), 3.73 (s, 3H), 3.58 (t, J=4.54 Hz, 4H), 2.59 (t, J=4.56 Hz, 4H).

### Methyl 4-(3-amino-2-hydroxybenzyl)piperazine-1-carboxylate (2)

To a solution of methyl 4-(2-hydroxy-3-nitrobenzyl)piperazine-1-carboxylate (1.0 equivalents) in 1:1 mixture of THF and MeOH (25 mL) was added 10% Pd/C (150 mg), and the mixture was pressurized with H₂ gas balloon at RT. The reaction was stirred for 2 h under hydrogen, the reaction mixture was filtered through a pad of celite. The resulting solution was concentrated in vacuo to provide the aniline as a brown solid, which was used in the next step without further purification (100% yield). ¹H NMR (CDCl₃, 400 MHz) δ: 7.06 (t, J=7.71 Hz, 1H), 6.89 (d, J=7.08 Hz 1H) 6.73 (d, J=7.83 Hz, 1H), 4.11 (s, 2H), 3.71 (s, 3H), 3.65 (s, 2H) 3.53 (s, 4H), 2.53 (s, 4H);

### Methyl 4-(2-hydroxy-3-(3-(6-methylpyridin-3-yl)ureido)benzyl)piperazine-1-carboxylate (3)

To a solution of Methyl 4-(3-amino-2-hydroxybenzyl)piperazine-1-carboxylate (1 equivalents) 2 in THF (5 volumes) was added O-benzoyl-N-(6-methylpyridin-3-yl)hydroxylamine followed by acetonitrile (5 volumes) to the above slurry DIPEA was added drop wise and heated to 55-60° C. for 16 h. After completion of reaction solvent was evaporated and residue was purified by flash column chromatography to get desired compound 3 in 70% yield.). ¹H NMR (CDCl₃, 400 MHz) δ 8.42 (d, J=2.19 Hz, 1H), 8.37 (s, 1H), 8.11 (dd, J=3.60 Hz, 1H), 8.05 (d, J=7.98 Hz, 1H), 7.69 (s, 1H), 7.14 (d, J=8.52 Hz, 1H), 6.80 (t, J=7.83 Hz, 1H), 6.68 (d, J=7.38 Hz, 1H), 3.75 (s, 3H), 3.73 (s, 2H), 3.56 (s, 4H). 2.58 (s, 3H), 2.53 (s, 4H).

### Methyl 4-(3-(3-(6-methylpyridin-3-yl)ureido)-2-(tosyloxy)benzyl)piperazine-1-carboxylate (4a)

To a solution of Methyl 4-(2-hydroxy-3-(3-(6-methylpyridin-3-yl)ureido)benzyl)piperazine-1-carboxylate (1 equivalents) in DMF (2 volumes) potassium carbonate (1.2 equivalents) was added and followed by tosyl chloride (1.2 equivalents) and stirred overnight, evaporated the solvent and residue was purified by column to get tosyl analog 4a in 62% yield.). ¹H NMR (CDCl₃, 400 MHz) δ: 8.30 (s, 1H), 7.92 (dd, J=3.45 Hz, 1H), 7.76 (d, J=7.80 Hz, 1H), 7.70 (d, J=7.98 Hz, 1H), 7.63 (s, 1H), 7.47 (s, 1H), 7.21 (m, 3H), 7.16 (m, 1H), 7.08 (d, J=8.46 Hz, 1H), 3.65 (s, 3 h), 3.43 (s, 2H), 3.39 (s, 4H), 2.47 (s, 3H), 2.29 (s, 4H), 2.20 (s, 3H).

### Methyl 4-(3-(3-(6-methylpyridin-3-yl)ureido)-2-(trifluoromethylsulfonyloxy)benzyl)piperazine-1-carboxylate (4b)

An oven-dried Schlenk tube equipped with a magnetic stirring bar under argon atmosphere was charged with 3 (1 equiv). Anhydrous dichloromethane (6 M) and NEt3 (1.5 equiv) was added before slow addition of trifluoromethanesulfonic anhydride (1.2 equiv) at 0° C. Then the solution was stirred at room temperature during the night. After extraction (from CH₂Cl2/H₂O), the organic layer was removed in vacuum. The crude product was purified by column chromatography on silica or crystallized (heptane/ethyl acetate), the corresponding aryl triflate was isolated in 70% yield. ¹H NMR (CDCl₃, 400 MHz) δ: 8.41 (s, 1H), 8.27 (s, 1H), 8.08 (m, 2H), 7.65 (s, 1H), 7.14 (d, J=8.49 Hz, 1H)), 6.81 (t, J=7.83 Hz, 1H), 6.68 (d, J=7.50 Hz), 3.75 (s, 3H), 3.73 (s, 2H), 3.56 (s, 4H), 2.57 (s, 3H), 2.53 (s, 4H).

### Methyl 4-(2-(2-hydroxyethoxy)-3-(3-(6-methylpyridin-3-yl)ureido)benzyl)piperazine-1-carboxylate (5)

To a solution of Methyl 4-(2-hydroxy-3-(3-(6-methylpyridin-3-yl)ureido)benzyl)piperazine-1-carboxylate (1 equivalents) in DMF (2 volumes) potassium carbonate (1.2 equivalents) was added and followed by 2-bromo ethanol (1.2 equivalents) and heated to 50° C. for 15-20 h. reaction mixture cooled to RT and poured in ice-water and extracted with ethyl acetate, separated layers organic was dried with sodium sulfate ethyl acetate was evaporated and residue was purified by column to get desired compound 4 in 38% yield. ¹H NMR (DMSO-d₆, 400 MHz) δ: 9.21 (s, 1H), 8.47 (s, 1H), 8.36 (s, 1H), 7.98 (d, J=7.71 Hz, 1H), 7.83 (dd, J=3.43 Hz, 1H), 7.17 (d, J=8.37 Hz, 1H), 7.04 (t, J=7.74 Hz, 1H), 6.98 (d, J=7.23 Hz, 1H), 5.21 (s, 1H), 3.92 (m, 2H), 3.77 (s, 2H), 3.35 (s, 3H), 3.55 (2.09), 2.40 (bs, 8H).

### Methyl 4-(3-(3-(6-methylpyridin-3-yl)ureido)-2-(2-(tosyloxy)ethoxy)benzyl)piperazine-1-carboxylate (6)

To a solution of Methyl 4-(2-(2-hydroxyethoxy)-3-(3-(6-methylpyridin-3-yl)ureido)benzyl)piperazine-1-carboxylate (1 equivalents) in dry THF (5 volumes) NaH was added and stirred for 10 min. Tosyl chloride was dissolved in dry THF and added to above NaH solution under nitrogen. Stirred for 1 h and evaporated and purified by flash column chromatography to get desired compound in 42% yield. ¹H NMR (DMSO-d₆, 400 MHz) δ: 9.45 (s, 1H), 8.67 (s, 1H), 8.48 (d, J=2.58 Hz, 1H), 8.29 (dd, J=3.27 Hz, 1H), 7.95 (s, 1H), 7.83 (dd, J=3.68 Hz, 1H), 7.47 (d, J=8.04 Hz, 2H), 7.14 (m, 5H), 4.81 (s, 2H), 4.45 (bs, 2H), 4.04 (bs, 2H), 3.75 (bs, 4H), 3.66 (s, 3H), 3.52 (bs, 4H), 2.89 (s, 3H), 2.73 (s, 3H), 2.40 (s, 3H), 2.28 (s, 3H); ¹³C NMR (100 MHz, CDCl₃) δ: 160.3, 152.9, 150.4, 145.5, 137.1, 135.60, 130.1, 126.0, 123.8, 123.5, 120.8, 118.9, 97.57, 50.9, 33.8, 18.8.

### Synthesis of VDA-28 (Radio Labeling)

### Methyl 4-(2-(2-fluoroethoxy)-3-(3-(6-methylpyridin-3-yl)ureido)benzyl)piperazine-1-carboxylate

To a solution of 2 mCi ¹⁸F in water was added 4.6 mg of K₂CO₃ and 25 mg of kryptofix heat to 90° C. under nitrogen and dried after 10 min, added ACN and evaporated again repeated same for 2-3 times to make sure of dryness. Compound 5 (1 mg) was dissolved in anhydrous DMF (200 μL) heated for 10 min, reaction mixture was injected to PREP-HPLC and collected radioactive compound fraction and formulated for study.

The present disclosure is directed to at least the following non-limiting illustrative embodiments.

Illustrative Embodiment 1: In at least one embodiment, the present disclosure is directed to a cardiac myosin targeting vector, comprising: a head, and a lipid anchoring/solubilizing moiety conjugated to the head via a linker molecule, wherein the head comprises omecamtiv mecarbil (OM) or an OM analog or derivative selected from the group consisting of:

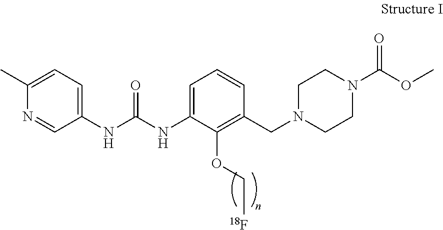

where n=2-10,

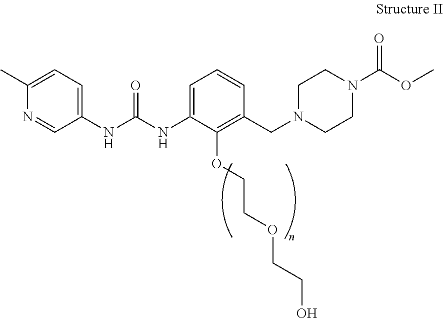

where n=1-2000, and

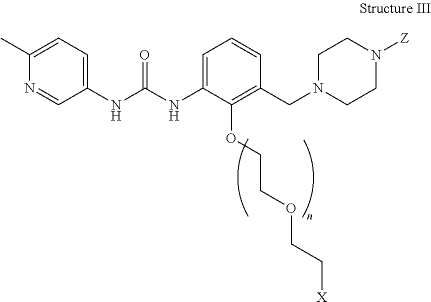

where
n=1-2000,
X=OH, OCH₃, F, ¹⁸F, and NH₂, and
Z=CH₃; acetyl; carbamoyl; —(CH₂)ₚCH₃, where p=1-20; or HO—(CH₂CH₂O)_{q}—H, where q=2-2,000.

Illustrative Embodiment 2: The cardiac myosin targeting vector of Illustrative Embodiment 1, wherein the linker molecule comprises a polyethylene glycol (PEG) molecule.

Illustrative Embodiment 3: The cardiac myosin targeting vector of Illustrative Embodiments 1 or 2, comprising a cargo molecule linked thereto.

Illustrative Embodiment 4: The cardiac myosin targeting vector of Illustrative Embodiment 3, wherein the cargo molecule is a therapeutic drug or an imaging agent.

Illustrative Embodiment 5: The cardiac myosin targeting vector of any one of Illustrative Embodiments 1-4 complexed with or conjugated to ⁹⁹Tc.

Illustrative Embodiment 6: The cardiac myosin targeting vector of Illustrative Embodiment 1 disposed in a liposomal carrier, wherein the liposomal carrier contains a cargo molecule, and wherein the anchoring/stabilizing moiety of the cardiac myosin targeting vector is anchored within the liposomal carrier, and the head of the cardiac myosin targeting vector extends outwardly from the liposomal carrier.

Illustrative Embodiment 7: The cardiac myosin targeting vector of Illustrative Embodiment 6, wherein the cargo molecule is a therapeutic drug or an imaging agent.

Illustrative Embodiment 8: The cardiac myosin targeting vector of Illustrative Embodiments 6 or 7, wherein the linker molecule of the cardiac myosin targeting vector comprises a polyethylene glycol (PEG) molecule.

Illustrative Embodiment 9: An omecamtiv mecarbil (OM) analog or derivative, comprising a structure selected from the group consisting of:

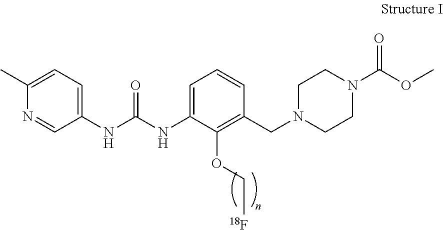

where n=2-10,

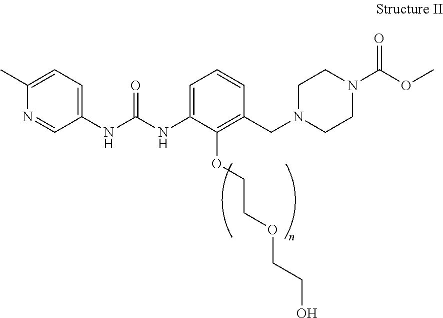

where n=1-2000, and

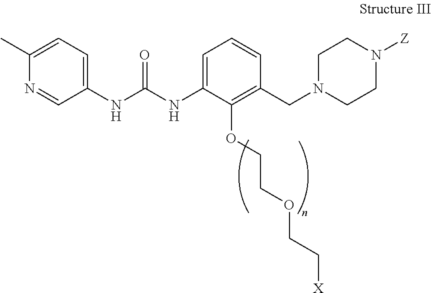

where
n=1-2000,
X=OH, OCH₃, F, ¹⁸F, and NH₂, and
Z=CH₃; acetyl; carbamoyl; —(CH₂)ₚCH₃, where p=1-20; or HO—(CH₂CH₂O)_{q}—H, where q=2-2,000.

Illustrative Embodiment 10: The OM analog or derivative of Illustrative Embodiment 9, further comprising a cargo molecule linked thereto.

Illustrative Embodiment 11: The OM analog or derivative of Illustrative Embodiment 9 or 10 complexed with or conjugated to ⁹⁹Tc.

Illustrative Embodiment 12: The OM analog or derivative of any one of Illustrative Embodiments 9-11 disposed within a liposomal carrier.

Illustrative Embodiment 13: A method of treating a cardiac condition or disease in a subject in need of such therapy, comprising administering to the subject (1) a cardiac-treating drug and (2) a cardiac myosin targeting vector, wherein the cardiac myosin targeting vector comprises: a head, and a lipid anchoring/solubilizing moiety conjugated to the head via a linker molecule, wherein the head comprises omecamtiv mecarbil (OM) or an OM analog or derivative selected from the group consisting of

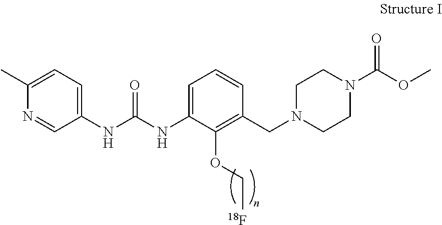

where n=2-10,

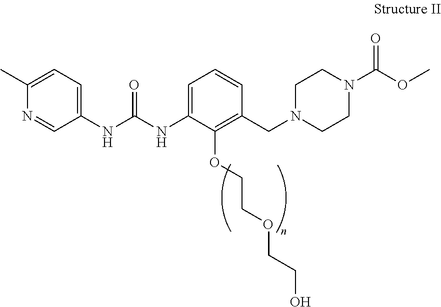

where n=1-2000, and

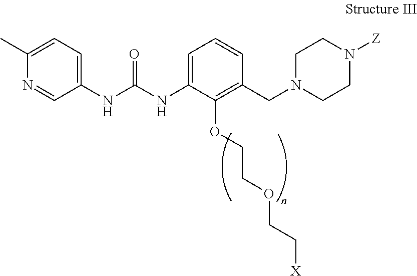

where
n=1-2000,
X=OH, OCH₃, F, ¹⁸F, and NH₂, and
Z=CH₃; acetyl; carbamoyl; —(CH₂)ₚCH₃, where p=1-20; or HO—(CH₂CH₂O)_{q}—H, where q=2-2,000.

Illustrative Embodiment 14: The method of Illustrative Embodiment 13, wherein the cardiac-treating drug is linked to the cardiac myosin targeting vector.

Illustrative Embodiment 15: The method of Illustrative Embodiment 13 or 14, wherein the cardiac-treating drug and the cardiac myosin targeting vector are combined in a liposomal carrier, wherein the liposomal carrier is decorated with the cardiac myosin targeting vector such that the anchoring/stabilizing moiety of the cardiac myosin targeting vector extends into an interior of the liposomal carrier, and the head of the cardiac myosin targeting vector extends outwardly from the liposomal carrier.

Illustrative Embodiment 16: The method of any one of Illustrative Embodiments 13-15, wherein the cardiac condition or disease is selected from the group consisting of a myocardial infarction, myocardial ischemia, reperfusion injury, congestive heart failure (CHF), a cardiomyopathy, coronary artery disease (CAD), atrial fibrillation, inflammation, atherosclerosis, unstable angina, an arrhythmia, a valve disease, a congenital or inherited heart condition, and a heart infection.

Illustrative Embodiment 17: A method of imaging a heart in a subject in need of such treatment, comprising administering to the subject an ¹⁸F-labeled analog of omecamtiv mecarbil, and imaging the heart of the subject using positron emission tomographic (PET) imaging.

Illustrative Embodiment 18: The method of Illustrative Embodiment 17, wherein the subject previously experienced or is currently experiencing a cardiac condition or disease.

Illustrative Embodiment 19: The method of Illustrative Embodiment 18, wherein the cardiac condition or disease is selected from the group consisting of a myocardial infarction, myocardial ischemia, reperfusion injury, congestive heart failure (CHF), a cardiomyopathy, coronary artery disease (CAD), atrial fibrillation, inflammation, atherosclerosis, unstable angina, an arrhythmia, a valve disease, a congenital or inherited heart condition, and a heart infection.

Illustrative Embodiment 20: The method of any one of Illustrative Embodiments 17-19, wherein the ¹⁸F-labeled analog of omecamtiv mecarbil has the structure:

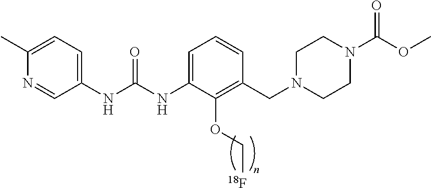

where n=2-10, or

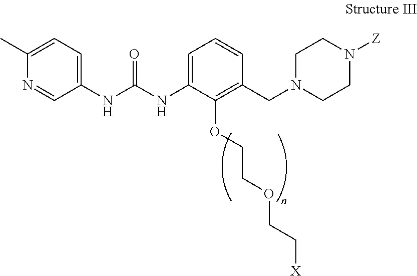

where
n=1-2000,
X=¹⁸F, and
Z=CH₃; acetyl; carbamoyl; —(CH₂)ₚCH₃, where p=1-20; or HO—(CH₂CH₂O)_{q}—H, where q=2-2,000.

Illustrative Embodiment 21: A method of synthesizing an ¹⁸F-labeled analog of omecamtiv mecarbil, comprising the steps of:
providing a compound having chemical structure 2, as represented in Scheme I;
reacting the compound having chemical structure 2 to form a compound having chemical structure 4, as represented in Scheme I;
reacting the compound having chemical structure 4 to form a compound having chemical structure 5, as represented in Scheme I, wherein TsO represents a tosyl group; and
reacting the compound having chemical structure 5 with an ¹⁸F donor to form a compound having chemical structure ¹⁸F-OMA, as represented in Scheme I,

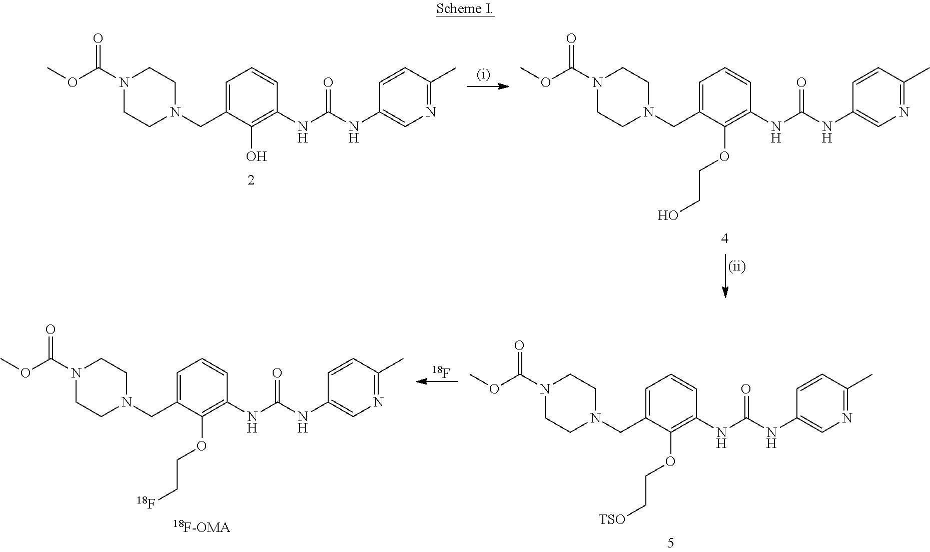

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure. Changes may be made in the formulations of the various compositions described herein, the methods described herein or in the steps or the sequence of steps of the methods described herein without departing from the spirit and scope of the present disclosure. Further, while various embodiments of the present disclosure have been described in exemplary claims herein below, it is not intended that the present disclosure be limited to these particular exemplary claims.

What is claimed is:

1. A cardiac myosin targeting vector, comprising: a head, and a lipid anchoring/solubilizing moiety conjugated to the head via a linker molecule, wherein the head comprises omecamtiv mecarbil (OM) or an OM analog or derivative selected from the group consisting of: 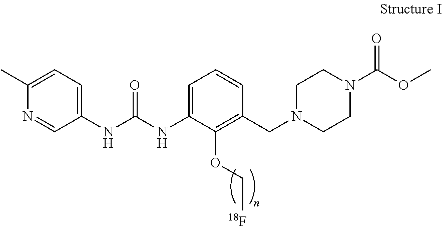
where n=2-10, 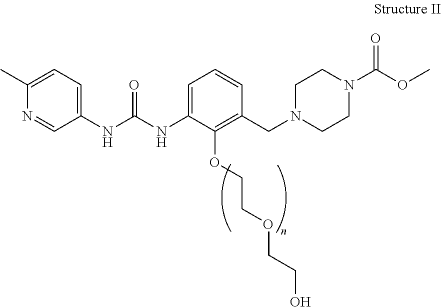
where n=1-2000, and 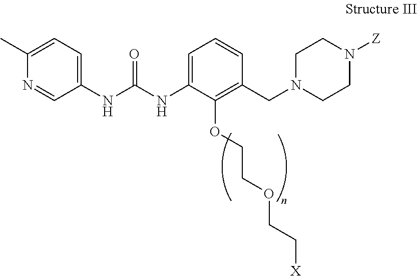
where
n=1-2000,
X=OH, OCH₃, F, ¹⁸F, and NH₂, and
Z=CH₃; acetyl; carbamoyl; —(CH₂)ₚCH₃, where p=1-20; or HO—(CH₂CH₂O)_{q}—H, where q=2-2,000.

2. The cardiac myosin targeting vector of claim 1, wherein the linker molecule comprises a polyethylene glycol (PEG) molecule.

3. The cardiac myosin targeting vector of claim 1, comprising a cargo molecule linked thereto.

4. The cardiac myosin targeting vector of claim 3, wherein the cargo molecule is a therapeutic drug or an imaging agent.

5. The cardiac myosin targeting vector of claim 1 complexed with or conjugated to ⁹⁹Tc.

6. The cardiac myosin targeting vector of claim 1 disposed in a liposomal carrier, wherein the liposoma carrier contains a cargo molecule, and wherein the anchoring/stabilizing moiety of the cardiac myosin targeting vector is anchored within the liposomal carrier, and the head of the cardiac myosin targeting vector extends outwardly from the liposomal carrier.

7. The cardiac myosin targeting vector of claim 6, wherein the cargo molecule is a therapeutic drug or an imaging agent.

8. The cardiac myosin targeting vector of claim 6, wherein the linker molecule of the cardiac myosin targeting vector comprises a polyethylene glycol (PEG) molecule.

9. An omecamtiv mecarbil (OM) analog or derivative, comprising a structure selected from the group consisting of: 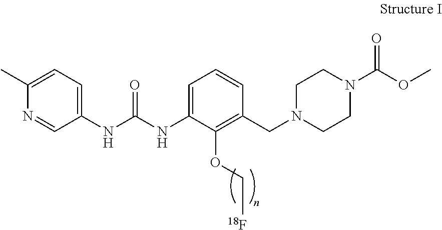
where n=2-10, 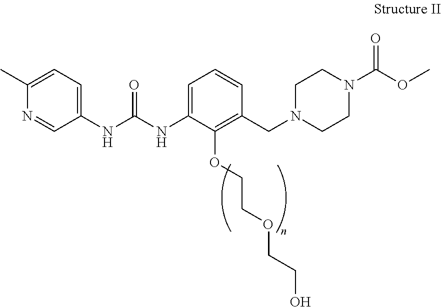
where n=1-2000, and 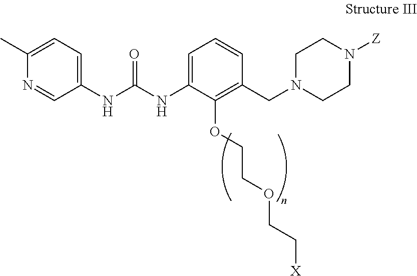
where
n=1-2000,
X=OH, OCH₃, F, ¹⁸F, and NH₂, and
Z=CH₃; acetyl; carbamoyl; —(CH₂)ₚCH₃, where p=1-20; or HO—(CH₂CH₂O)_{q}—H, where q=2-2,000.

10. The OM analog or derivative of claim 9, further comprising a cargo molecule linked thereto.

11. The OM analog or derivative of claim 9 complexed with or conjugated to ⁹⁹Tc.

12. The OM analog or derivative of claim 9 disposed within a liposomal carrier.

13. A method of treating a cardiac condition or disease in a subject in need of such therapy, comprising administering to the subject (1) a cardiac-treating drug and (2) a cardiac myosin targeting vector, wherein the cardiac myosin targeting vector comprises: a head, and a lipid anchoring/solubilizing moiety conjugated to the head via a linker molecule, wherein the head comprises omecamtiv mecarbil (OM) or an OM analog or derivative selected from the group consisting of: 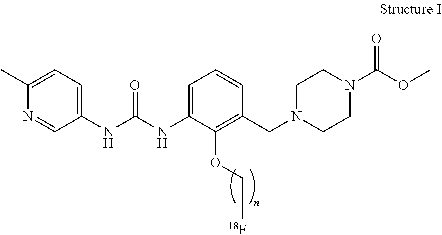
where n=2-10, 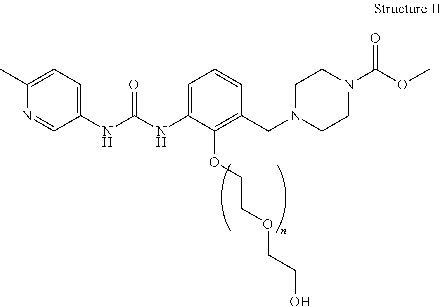
where n=1-2000, and 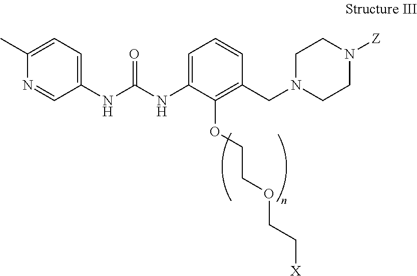
where
n=1-2000,
X=OH, OCH₃, F, ¹⁸F, and NH₂, and
Z=CH₃; acetyl; carbamoyl; —(CH₂)ₚCH₃, where p=1-20; or HO—(CH₂CH₂O)_{q}—H, where q=2-2,000.

14. The method of claim 13, wherein the cardiac-treating drug is linked to the cardiac myosin targeting vector.

15. The method of claim 13, wherein the cardiac-treating drug and the cardiac myosin targeting vector are combined in a liposomal carrier, wherein the liposomal carrier is decorated with the cardiac myosin targeting vector such that the anchoring/stabilizing moiety of the cardiac myosin targeting vector extends into an interior of the liposomal carrier, and the head of the cardiac myosin targeting vector extends outwardly from the liposomal carrier.

16. The method of claim 13, wherein the cardiac condition or disease is selected from the group consisting of a myocardial infarction, myocardial ischemia, reperfusion injury, congestive heart failure (CHF), a cardiomyopathy, coronary artery disease (CAD), atrial fibrillation, inflammation, atherosclerosis, unstable angina, an arrhythmia, a valve disease, a congenital or inherited heart condition, and a heart infection.
